(19) United States
(12) Patent Application Publication
(10) Pub. No.: US 2024/0009400 A1
(43) Pub. Date: Jan. 11, 2024

(54) STERILE SAMPLING METHODS AND DEVICES FOR AUTOMATED CELL ENGINEERING SYSTEMS

(71) Applicants: OCTANE BIOTECH INC., Kingston, Ontario (CA); LONZA WALKERSVILLE, INC., Walkersville, MD (US)

(72) Inventors: Chase MCROBIE, Kingston, Ontario (CA); Raelyn DANIELS, Kingston, Ontario (CA); Matthew HEWITT, Walkersville, MD (US)

(21) Appl. No.: 18/247,552

(22) PCT Filed: Oct. 8, 2021
(86) PCT No.: PCT/US2021/071787

§ 371 Date: Mar. 31, 2023

Related U.S. Application Data
(60) Provisional application No. 63/089,335, filed on Oct. 8, 2020.

(51) Int. Cl. A61M 5/315

(52) U.S. Cl. CPC A61M 5/31513

(57) ABSTRACT

Devices and methods for sterile sampling from automated cell engineering systems are provided. Sterile sampling devices are configured to maintain sterility of a sample reservoir during intake and expulsion of fluids or other material to the sterile sampling devices. Methods provided herein employ sterile sampling devices to achieve the sterile withdrawal and sterile injection of materials and fluids to and from an automated cell engineering system.

### FIELD OF THE INVENTION

The present disclosure is related to use of automated cell engineering systems. In particular, the present disclosure relates to methods and devices providing for sterile sampling from automated cell engineering systems and other sterile environments.

### BACKGROUND OF THE INVENTION

As anticipation builds about accelerated clinical adoption of advanced cell therapies, more attention is turning to the underlying manufacturing strategies that will allow these therapies to benefit patients worldwide.

The production of cells for cell therapies may require significant manual involvement due to the patient-specific product. In just one example, Automation of CAR T cell culture is particularly challenging due to the multiple sensitive unit operations, including cell activation, transduction and expansion.

Integration of cell activation, transduction and expansion into a commercial manufacturing platform is critical for the translation of these important immunotherapies to the broad patient population. For these life-saving treatments to be applicable to the global patient population, a shift in manufacturing techniques must be implemented to support personalized medicine. The benefits of automation have previously been described. These benefits include labor time savings associated with using automation as well as improved product consistency, decreased room classification, decreased clean room footprint, decreased training complexities, and improved scale-up and tracking logistics.

The benefits of automation may not be fully realized without appropriate sterile process control. The present application provides solutions for maintaining sterility during operations associated with automated cell engineering systems. Solutions provided herein are further suitable for maintaining sterility during operations that involve any type of sterile system.

### SUMMARY OF THE INVENTION

In embodiments, a sterile plunger syringe is provided. The sterile plunger syringe includes a syringe barrel defining a syringe reservoir and having an interconnect at a distal end and an opening surrounded by a syringe barrel flange at a proximal end; a syringe plunger including a syringe plunger rod flange, a plunger rod, and a reservoir face; a gasket disposed over the reservoir face and configured to provide a seal between the reservoir face and the syringe reservoir when the syringe plunger is seated within the syringe barrel; and a syringe plunger sealing device secured to the syringe barrel and configured to provide a syringe plunger seal.

In further embodiments, a method of sterile sampling from an automated cell engineering system is provided. The method includes providing a sterile plunger syringe including a syringe barrel, a syringe plunger, and a syringe plunger sealing device providing a syringe plunger seal; connecting the sterile plunger syringe to the automated cell engineering system; and withdrawing a biological sample from the automated cell engineering system with the sterile plunger syringe.

In further embodiments, a method of sterile sampling from an automated cell engineering system is provided. The method includes providing a sterile sampling device including a sample reservoir, a sample chamber, a filling device, and a sterile sealing apparatus; connecting the sterile sampling device to the automated cell engineering system; and withdrawing a biological sample from the automated cell engineering system via the filling device.

### BRIEF DESCRIPTION OF THE FIGURES

FIG. 1 shows a generalized manufacturing process for a cell culture.

FIG. 2 shows a lab space containing exemplary cell engineering systems as described in embodiments herein.

FIG. 3 shows a cell culture production process that can be performed in a cell engineering system as described in embodiments herein.

FIGS. 4A-4C show an overview of an automated cell engineering system . FIG. 4A shows an automated cell engineering system in the closed configuration. FIG. 4B shows a Cassette that can be inserted into an automated cell engineering system. FIG. 4C shows an automated cell engineering system in the open configuration.

FIGS. 4D-4E show the location and orientation of a cell culture chamber utilized in an automated cell engineering system .

FIG. 4F shows a more detailed view of the cell culture chamber utilized in an automated cell engineering system.

FIG. 4G shows a process flow legend for an automated cell engineering system

FIGS. 5A-5E show another configuration of an automated cell engineering system as described in embodiments herein. FIG. 5A shows a disposable cassette that can be loaded into the automated cell engineering system. FIG. 5B shows an automated cell engineering system in the open configuration. FIG. 5C shows the cassette loaded into the automated cell engineering system. FIG. 5D shows the automated cell engineering system in a closed configuration. FIG. 5E shows a detailed view of a cassette for use with the automated cell engineering system.

FIG. 6 shows use of a syringe and a bag to sample from the cassette.

FIG. 7 illustrates a sterile sampling device consistent with embodiments hereof.

FIGS. 8A-8B illustrate a sterile sampling device consistent with embodiments hereof

FIGS. 9A-9C illustrate additional sterile sampling devices consistent with embodiments hereof

FIG. 10 illustrates a further sterile sampling device consistent with embodiments hereof

FIGS. 11A-11E illustrate a sterile sampling device consistent with embodiments hereof

FIG. 12 is a flow diagram illustrating a sterile sampling process consistent with embodiments hereof

FIGS. 13A-13I show an example of a sterile sampling process consistent with embodiments hereof.

### DETAILED DESCRIPTION OF THE INVENTION

The present disclosure provides systems and methods of maintaining sterile processes in conjunction with automated cell engineering systems. Automated cell engineering systems provide powerful tools for production of various engineered cells and tissues, as well as biological materials (e.g., proteins, peptides, antibodies, antibody fragment, etc.). Among the benefits of automated cell engineering systems as described herein is the use of sterile self-contained modules. Such modules create a sterile environment for cell processing without the requirement of conducting such processes in a high-level clean room. When automated cell engineering systems are operated in non-sterile environments, steps must be taken to maintain sterility during operator interactions with these systems.

Methods and devices provided herein facilitate sterile sampling from automated cell engineering systems and therefore permit the removal and reinsertion of samples, e.g., cell cultures, biological material samples, reagent samples, and any other fluid or material samples, from an automated cell engineering system.

One automated cell engineering system consistent with embodiments hereof is the Cocoon198 platform, aspects of which are described in greater detail below. The Cocoon' platform is described in fuller detail in U.S. patent application Ser. No. 16/119,618, filed on Sep. 1, 2017, the contents of which are incorporated by reference herein in their entirety. Descriptions provided herein of specific systems or automated cell engineering systems that may be used with the sterile sampling devices and methods disclosed herein are by way of example only. The sterile sampling devices and methods disclosed herein may be applied to additional systems, for example, the ADVA_X3® (Adva Biotechnology) and CLINIMACS PRODIGY® (Miltenyi Biotech). In particular, the sterile sampling devices and methods disclosed herein may be suitably applied to any sterile system from which a sample is to be drawn while maintaining sterility of both the sample and the system. Further, the sterile sampling devices and methods disclosed herein may be suitably applied to any sterile system situated in a non-sterile environment.

As described herein, installation and comprehensive validation of automated manufacturing provides a solution to logistical and operational challenges for production of engineered cells and tissues. An important approach to introducing automation to a production process is identifying the key modular steps where the operator applies a physical, biological, or chemical change to the production material, termed “unit operations.” In the case of cell manufacturing, this includes steps such as cell separation, genetic manipulation, proliferation, washing, concentration, and cell harvesting. Manufacturers often identify local process bottlenecks as the immediate opportunities for introducing automation. This is reflected in the technical operation spectrum of the majority of commercially available bioreactors, which tend to focus on discrete process steps. Process challenges in cell manufacturing (from sterility maintenance to sample tracking) are addressed herein by end-to-end automation that generates consistent cellular outputs while ameliorating inevitable process variability. The methods described herein also provide simplification, and the associated electronic records aid in complying with GMP standards.

The recent rapid progress of the clinical development of various cell cultures, including modified autologous T cells for cancer immunotherapy, has led to planning for the associated translation and scale up/out implications.

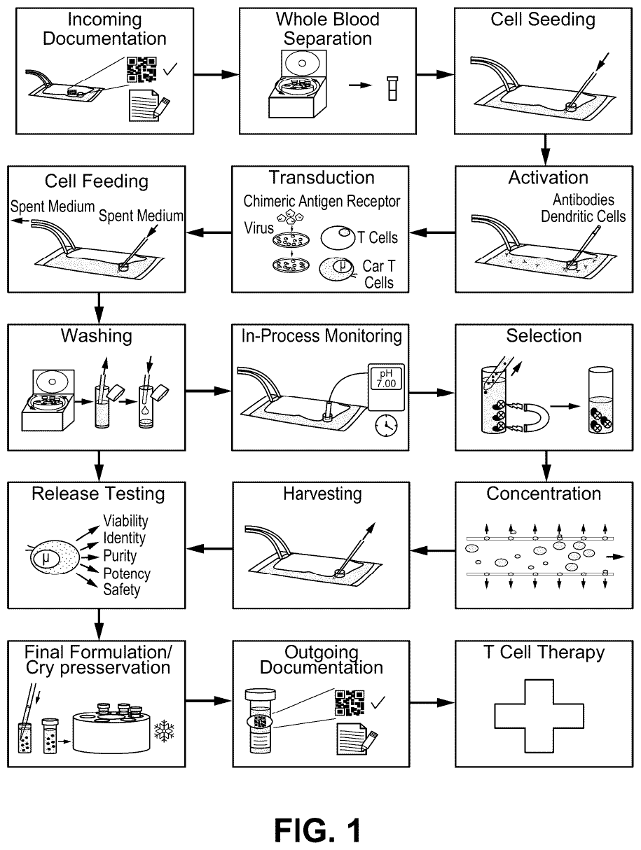

While specific cell culture growth protocols may vary for cell manufacturing, a generalized cell culture production process is illustrated in FIG. 1 (including production of autologous T cells). FIG. 1 describes unit operations of cell manufacturing, e.g., from initial processing of a patient blood sample to formulating output cells for autologous T cell therapy.

As described herein, to achieve cell manufacturing automation, the sampling methods described herein provide for understanding the status of the cells at each transition point and how they are impacted by the specific unit operation through the use of external analysis equipment. The micro-lot production for patient-specific therapies should be respectful of key process sensitivities that impact the feasibility of automation. Automation described herein successfully embraces various process steps.

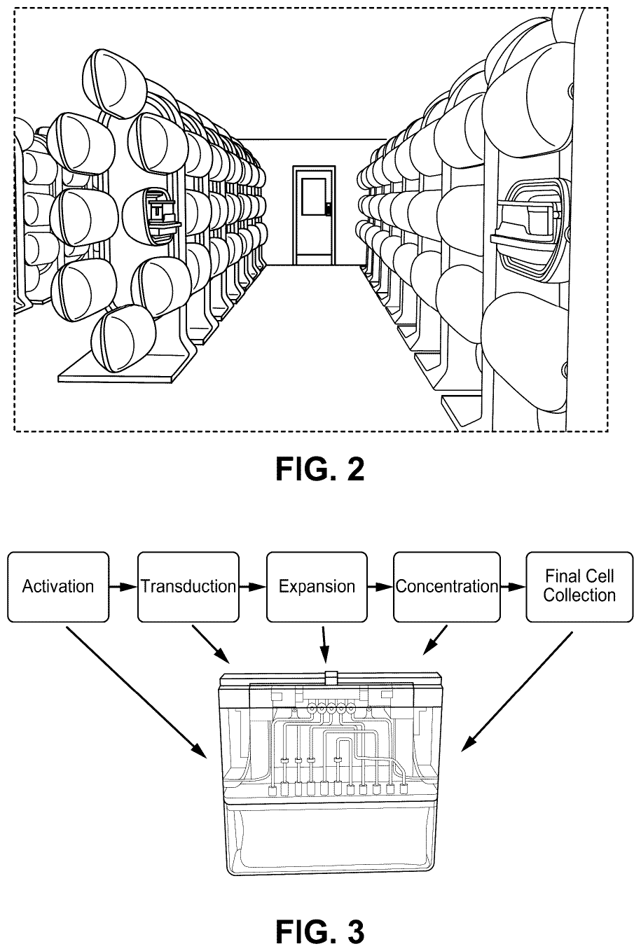

A single all-in-one system can offer significantly greater space efficiency to minimize the required footprint in expensive GMP clean rooms. For example, as shown in FIG. 2, fully integrated automated systems are designed to maximize required footprint to reduce expensive GMP clean room space. FIG. 2 shows e.g., 96 patient-specific end-to-end units running in a standard lab space.

As described herein, in embodiments, the methods provided utilize the COCOON platform (Octane Biotech (Kingston, ON)), which integrates multiple unit operations in a single turnkey platform (see e.g., U.S. Published Patent Application No. 2019/0169572, the disclosure of which is incorporated by reference herein in its entirety). It is understood, however, that other fully or partially automated cell culture apparatus may be used according to embodiments hereof, including those commercially available such as PRODIGY available from Miltenyi Biotech, Inc., XURI and SEFIA from General Electric Healthcare, and systems available from Atvio Biotech Ltd. The sterile sampling devices and methods described herein may be suitable for sterile sampling operations performed with each of the above-listed and any other commercial device.

The methods described herein can be used in conjunction with the production of CAR T cells (including activation, viral transduction and expansion, concentration and washing) in a fully-integrated closed automation system (FIG. 3).

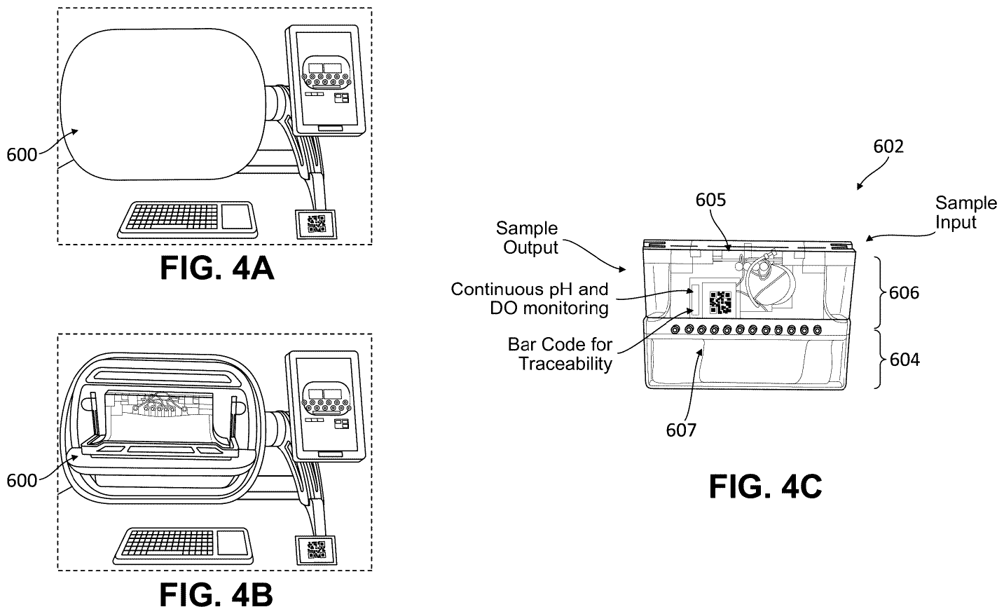

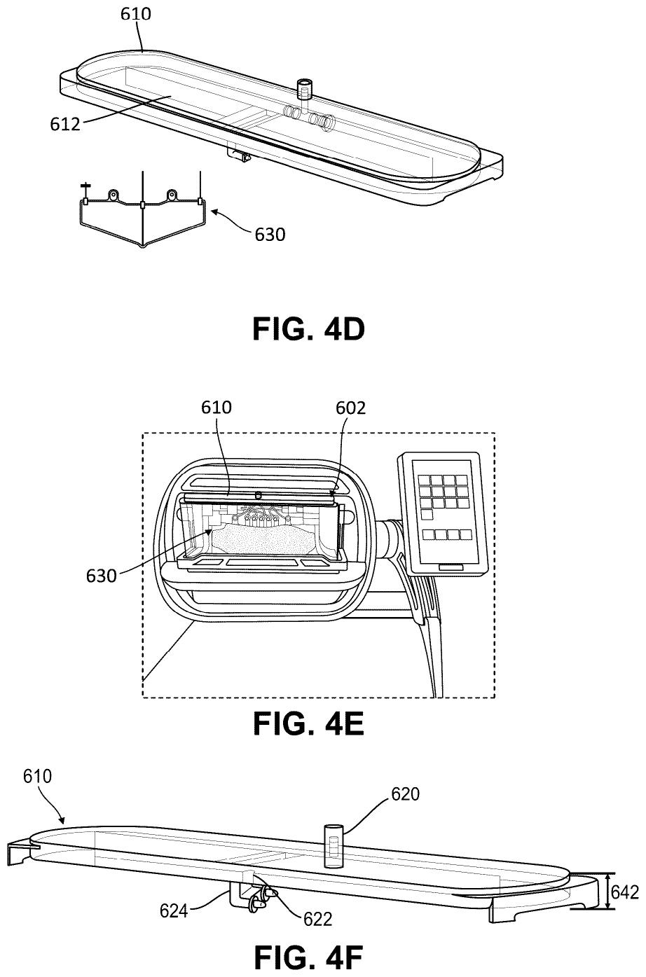

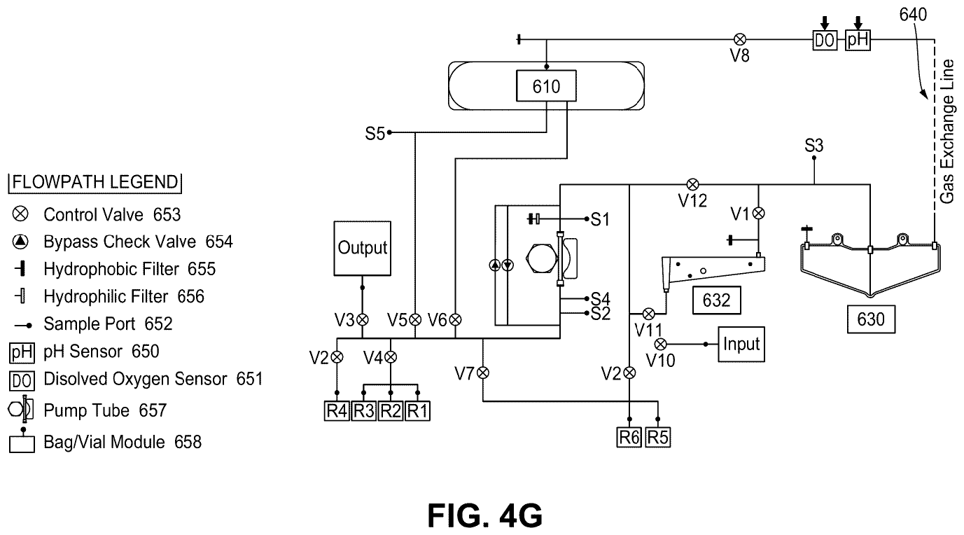

In some embodiments, the methods described herein are performed in connection with a functionally enclosed, automated cell engineering system 600 (see FIGS. 4A, 4B), suitably having instructions thereon for performing activating, transducing, expanding, concentrating, and harvesting steps, of cell cultures. Cell engineering systems (also called automated cell engineering systems throughout) provide for the automated production of cell cultures. As used herein “cell cultures” refers to any suitable cell type, including individual cells, as well as multiple cells or cells that may form into tissue structures. Exemplary cell cultures include blood cells, skin cells, muscle cells, bone cells, cells from various tissues and organs, etc. In embodiments, genetically modified immune cells, including CAR T cells, as described herein, can be produced. Exemplary automated cell engineering systems are also called COCOON, or COCOON system throughout.

For example, a user can provide a cell engineering system pre-filled with a cell culture and reagents (e.g., an activation reagent, a vector, cell culture media, nutrients, selection reagent, and the like) and parameters for the cell production (e.g., starting number of cells, type of media, type of activation reagent, type of vector, number of cells or doses to be produced, and the like), the cell engineering system is able to carry out methods of producing an engineering cell culture, including genetically modified immune cell cultures, including CAR T cells, without further input from the user. At the end of the automated production process, the cell engineering system may alert the user (e.g., by playing an alert message or sending a mobile app alert) for collecting the produced cells. In some embodiments, the functionally enclosed cell engineering system includes sterile cell culture chambers. Functionally enclosed refers to a system which is self-contained but may include means of gas exchange, for example, hydrophobic filters and gas permeable tubing. In some embodiments, the functionally enclosed cell engineering system minimizes contamination of the cell cultures by reducing exposure of the cell culture to non-sterile environments. In additional embodiments, the functionally enclosed cell engineering system minimizes contamination of the cell cultures by reducing user handling of the cells.

As described herein, the cell engineering systems suitably include a cassette 602 (see FIG. 4B). As used herein a “cassette” refers to a largely self-contained, removable and replaceable element of a cell engineering system that includes one or more chambers for carrying out the various elements of the methods described herein, and suitably also includes one or more of a cell media, an activation reagent, a vector, etc. A cassette can include a flexible bag, rigid container, or other construction element. In some aspects, the cassette can be configured for a single-use.

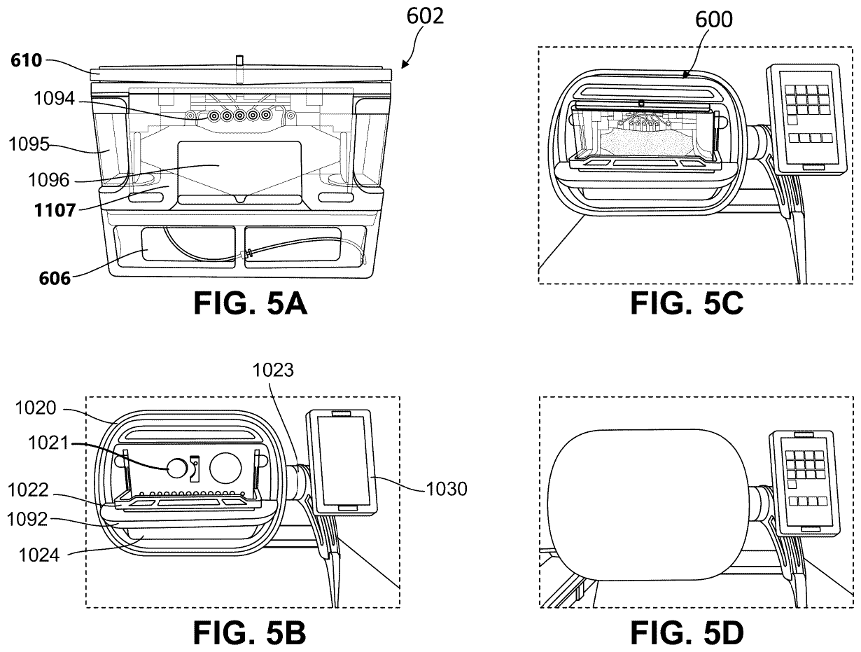

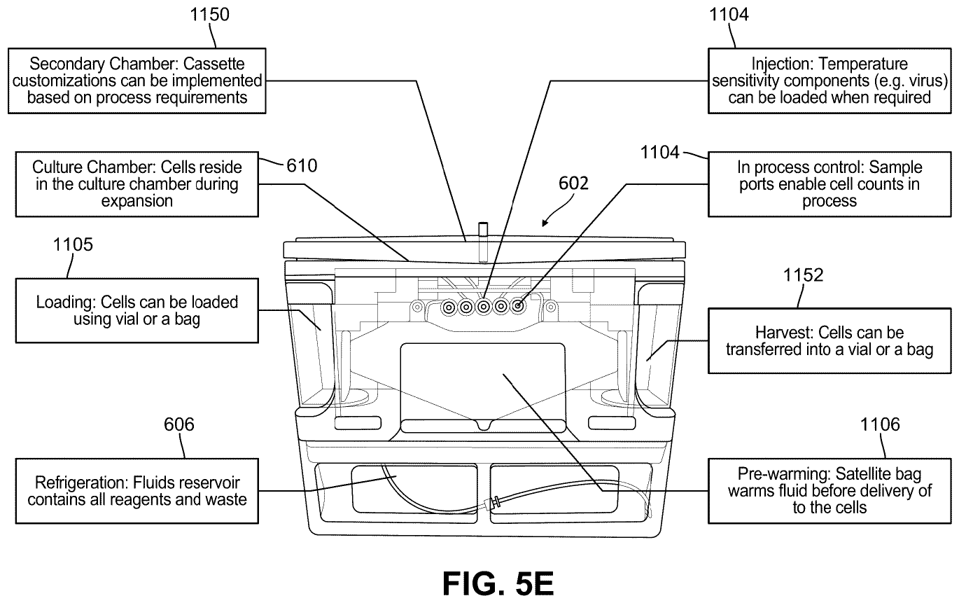

FIG. 4B shows an embodiments of a cassette 602 in accordance with embodiments hereof In embodiments, cassette 602 includes a low temperature chamber 604, suitably for storage of a cell culture media, as well as a high temperature chamber 606, suitably for carrying out activation, transduction and/or expansion of an immune cell culture. Suitably, high temperature chamber 606 is separated from low temperature chamber 604 by a thermal barrier 1092 (see FIG. 5b). As used herein “low temperature chamber” refers to a chamber, suitably maintained below room temperature, and more suitably from about 2° C. to about 8° C., for maintenance of cell media, etc., at a refrigerated temperature. The low temperature chamber can include a bag or other holder for media, including about 1 L, about 2 L, about 3 L, about 4 L, or about 5 L of fluid. Additional media bags or other fluid sources can be connected externally to the cassette and connected to the cassette via an access port, for example, closed Luer fittings, welded tubing, etc.

As used herein “high temperature chamber” refers to chamber, suitably maintained above room temperature, and more suitably maintained at a temperature to allow for cell proliferation and growth, i.e., between about 35-39° C., and more suitably about 37° C.

In embodiments, high temperature chamber 606 suitably includes a cell culture chamber 610 (also called proliferation chamber or cell proliferation chamber throughout), as shown in FIG. 4d and FIG. 4e.

The cassettes can, in some aspects, further include one or more fluidics pathways connected to the cell culture chamber, wherein the fluidics pathways provide recirculation, removal of waste and homogenous gas exchange and distribution of nutrients to the cell culture chamber without disturbing cells within the cell culture chamber. Cassette 602 also further includes one or more pumps 605, including peristaltic pumps, for driving fluid through the cassette, as described herein, as well as one or more valves 607, for controlling the flow through the various fluidic pathways.

In exemplary embodiments, as shown in FIG. 4d, cell culture chamber 610 is flat and non-flexible chamber (i.e., made of a substantially non-flexible material such as a plastic) that does not readily bend or flex. The use of a non-flexible chamber allows the cells to be maintained in a substantially undisturbed state. As shown in FIG. 4e, cell culture chamber 610 is oriented so as to allow the cell culture to spread across the bottom 612 of the cell culture chamber. As shown in FIG. 4e, cell culture chamber 610 is suitably maintained in a position that is parallel with the floor or table, maintaining the cell culture in an undisturbed state, allowing the cell culture to spread across a large area of the bottom 612 of the cell culture chamber. In embodiments, the cell culture chamber may include features, such as a warp, to facilitate consistent filling and draining. In embodiments, the overall thickness of cell culture chamber 610 (i.e., the chamber height 642) is low, on the order of about 0.5 cm to about 5 cm. As described herein, in exemplary embodiments the cassette is pre-filled with one or more of a cell culture, a culture media, an activation reagent, and/or a vector, including any combination of these. In further embodiments, these various elements can be added later via suitable injection ports, etc.

As described herein, in embodiments, the cassettes suitably further include one or more of a pH sensor, a glucose sensor, a dissolved oxygen sensor, a carbon dioxide sensor, a lactic acid sensor/monitor, and/or an optical density sensor. The cassettes can also include one or more sampling ports and/or injection ports. Examples of such sampling ports and injection ports (1094) are illustrated in FIG. 5a. and can include an access port for connecting the cartridge to an external device, such as an electroporation unit or an additional media source. FIG. 5a also shows the location of the cell input 1095, reagent warming bag 1096 which can be used to warm cell media, etc., as well as the culture zone 1107, which holds various components for use in the culture media, including for example, cell media, vectors, nutrients and waste products, etc.

FIG. 5b shows the COCOON cell engineering system with cassette 602 removed. Visible in FIG. 5b are components of the cell engineering system, including gas control seal 1020, warming zone 1021, actuators 1022, pivot 1023 for rocking or tilting the cell engineering system as desired, and low temperature zone 1024 for holding low temperature chamber 604. Also shown is an exemplary user interface 1030, which can include a reader for one and two dimensional codes, e.g., bar codes and QR codes, and the ability to receive using inputs by touch pad or other similar device. The user interface 1030 that may further include a component identification sensor such as a bar code reader, QR code reader, radio frequency ID interrogator, or other component identification sensor. In some aspects, a cassette 602 can include a first identification component, such as a bar code, and the user interface 1030 can include a reader that is configured to read and identify the first identification component. FIG. 5e shows an additional detailed view of cassette 602, including the location of secondary chamber 1150, which can be used is additional cell culture volume is required, as well as harvesting chamber 1152, which can be used to recover the final cell culture as produced herein.

In exemplary embodiments, as shown in FIG. 4f, cell culture chamber 610 further comprises at least one of: a distal port 620 configured to allow for the removal of air bubbles from the cell culture chamber and/or as a recirculation port; a medial port 622 configured to function as a recirculation inlet port; and a proximal port 624 configured to function as a drain port for cell removal.

In still further embodiments, provided herein is cassette 602 for use in an automated cell engineering system 600, comprising cell culture chamber 610 for carrying out activation, transduction and/or expansion of an immune cell culture having a chamber volume that is configured to house an immune cell culture and a satellite volume 630 for increasing the working volume of the cell culture chamber by providing additional volume for media and other working fluids without housing the immune cell culture (i.e., satellite volume does not contain any cells).

FIG. 4g shows a schematic illustrating the connection between cell culture chamber 610, and satellite volume 630. Also illustrated in FIG. 4g are the positioning of various sensors (e.g., pH sensor 650, dissolved oxygen sensor 651), as well as sampling/sample ports 652 and various valves (control valves 653, bypass check valves 654), as well as one or more fluidic pathways 640, suitably comprising a silicone-based or other tubing component, connecting the components. As described herein, use of a silicone-based tubing component allows oxygenation through the tubing component to facilitate gas transfer and optimal oxygenation for the cell culture. Also show in FIG. 4g is the use of one or more hydrophobic filters 655 or hydrophilic filters 656, in the flow path of the cassette, along with pump tube 657 and bag/valve module 658.

In additional embodiments, as shown in FIG. 4g, cassette 602 suitably further includes a crossflow reservoir 632 for holding additional media, etc., as needed. Suitably, the crossflow reservoir has a volume of between about 0.50 ml and about 300 ml, more suitably between about 100 ml and about 150 ml.

In some embodiments, the cell engineering system includes a plurality of chambers. In further embodiments, each of the activating, transducing, expanding, concentrating, and harvesting steps of the method for cells described herein is performed in a different chamber of the plurality of chambers of the cell engineering system. In some embodiments, the cells are substantially undisturbed during transfer from one chamber to another. In other embodiments, the steps of the method are performed in the same chamber of the cell engineering system, and the cell engineering system automatically adjusts the chamber environment as needed for each step of the method. Thus, further allowing for the cells to not be disturbed during the various steps.

Various processes and/or operations conducted with automated cell engineering systems may require withdrawing materials (such as cell cultures, biological material samples, or other fluids) from the automated cell engineering systems. Some processes and operations may further require injecting such materials back into the automated cell engineering systems. Additional requirements can include injection various materials into the systems, such as transfection reagents, as well as nutrient supplements, media supplements, etc. Such processes and operations may benefit from a sterile sampling device configured to draw and return a sample in a sterile fashion without introducing any contaminants from a non-sterile environment.

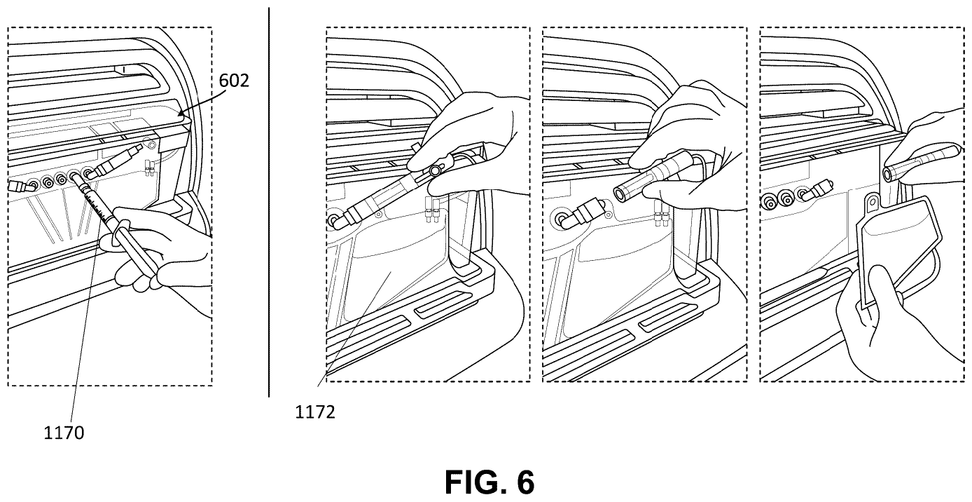

FIG. 6 shows the use of a conventional syringe 1170 and bag 1172 being used to withdraw a sample from an automated cell engineering system 600. Conventional syringes may be provided with sterile interiors. When operated in a non-sterile environment, i.e., an environment having less sterility than the sterile interior of the cell engineering system, it is possible for the sterility of the syringe interior to be compromised. For example, in a conventional plunger syringe, a dynamic seal is provided to permit sealing the syringe interior over the range of motion of the syringe plunger. However, a portion of the syringe interior behind the reservoir face is exposed to the environment during operation, i.e., when the syringe plunger is pressed in. Contaminants clinging to the walls of the interior have the potential to be introduced to any substances that are drawn into the syringe interior during operation of the plunger.

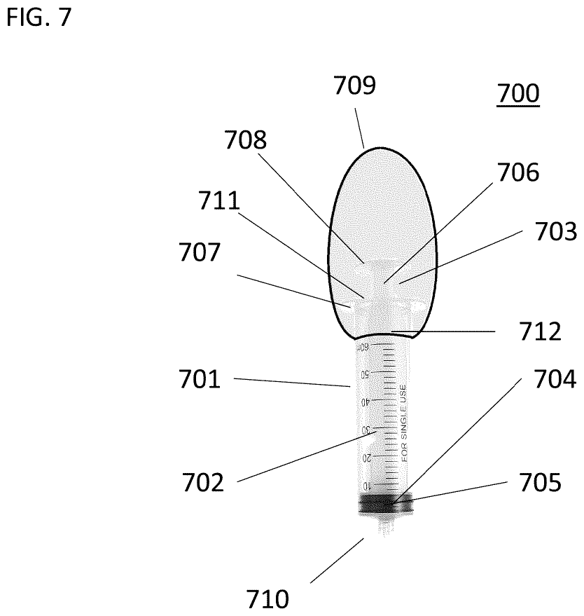

Specific embodiments related to sterile sampling devices and methods are now described with reference to FIGS. 7-13. Unless otherwise indicated, for the sterile sampling devices discussed herein, the terms “distal” and “proximal” are used in the following description with respect to a position or direction relative to the operator. “Distal” and “distally” are positions that refer to the input/output end of a sterile sampling device and may be distant from or in a direction away from the operator under standard use. “Proximal” and “proximally” are positions that refer to the operational end of a sterile sampling device, in a direction toward the clinician during normal use.

Embodiments herein provide a sterile sampling device suitable for maintaining sterility of a sample drawn into and/or expelled from the sterile sampling device. The sterile sampling device includes a sample chamber that defines a sample reservoir. The sample reservoir is configured to contain a sample, such as a biological material sample, a reagent sample, cell culture, or any other fluid. The sterile sampling device includes an interconnect at a distal end of the sample chamber. The interconnect is configured to connect the sample chamber to other sampling equipment, and may include, for example, weldable tubing, Luer activated connectors such as ICU Medical Spiros and BD Q-Syte, genderless connectors such as CPC AseptiQuik, standard Luer lock fittings, twist-fit, and any other suitable connector.

The sterile sampling device further includes a filling device configured, through manual or automated operation, to draw a sample into the sample reservoir and/or expel the sample from the sample reservoir. Filling devices consistent with embodiments herein include any device, mechanism, or system configured to provide a pressure differential (either over pressure or under pressure) between the sample reservoir as connected to a reservoir of the automated cell engineering system and an exterior environment. The pressure differential provided by the filling device drives drawing in and ejection of fluids from the sample reservoir. Filling devices are configured to connect to and operate with various sample chambers as discussed herein. For example, as discussed below, a syringe sample chamber may be employed with a syringe plunger as a filling device. Other sample chamber designs may employ a pump, vacuum, syringe, and other mechanisms as a filling device, as discussed below.

The sterile sampling device further includes a sterile sealing apparatus configured to maintain sterility of the sample reservoir from environmental contaminants during operation of the sterile sampling device, e.g., filling and emptying of the sample reservoir. As used herein, maintaining sterility of the sample reservoir refers to preventing contamination of the sample reservoir from the environment and maintaining the sample reservoir at a sterility level higher than that of the environment. The sterile sealing apparatus may or may not be liquid contacting.

Specific examples of various aspects of the sterile sampling device are discussed in greater detail below, with respect to FIGS. 7-11.

FIG. 7 illustrates a sterile sampling device consistent with embodiments hereof. The sterile sampling device of FIG. 7 is a sterile plunger syringe 700 and may include a syringe barrel 701, a syringe plunger 703, a gasket 705, and a syringe plunger sealing device 709.

The syringe barrel 701 is generally cylindrical and defines a syringe reservoir 702 that occupies the interior of the syringe barrel 701. The syringe barrel 701 is an example of a sample chamber defining a sample reservoir. A distal end of the syringe barrel 701 includes an interconnect 710. The interconnect 710 may be any type of syringe interconnect, including, for example, a Luer lock tip, a slip tip, an eccentric tip, and a catheter tip. Any suitable syringe tip may be employed as the interconnect 710. A proximal end of the syringe barrel 701 includes a proximal opening 711 surrounded by a syringe barrel flange 707. The proximal opening 711 has a diameter substantially the same as the syringe reservoir 702. The syringe barrel 701 may include graduated volume markings to indicate volume of a substance inside the syringe reservoir 702. The syringe barrel 701 may be manufactured of any suitable material, including, for example, polyethylene, polycarbonate, polypropylene, stainless steel, etc.

The syringe plunger 703 includes a syringe plunger rod 706, a syringe plunger rod flange 708 located at a proximal end of the syringe plunger rod 706, and a reservoir face 704 located at a distal end of the syringe plunger rod 706. The syringe plunger rod 706 may be manufactured of any suitable material, including, for example, polyethylene, polycarbonate, polypropylene, stainless steel, etc. The syringe plunger 703 is an example of a filling device configured to fill and empty the syringe reservoir 702.

The gasket 705 is disposed over the reservoir face 704 and is configured to provide a seal between the reservoir face 704 and the interior walls of the syringe barrel 701 when the syringe plunger 703 is seated within the syringe barrel 701. Thus, when the syringe plunger 703 is seated within the syringe barrel 701, the gasket 705 seals the syringe reservoir 702. When the sterile plunger syringe 700 is operated, the syringe plunger 703 is moved back and forth within the syringe reservoir 702 to intake and expel a fluid, i.e., a liquid or gas.

A portion of the syringe reservoir 702 distal of the reservoir face 704 is referred to herein as the active portion of the syringe reservoir 702 while a portion of the syringe reservoir 702 proximal of the reservoir face 704 is referred to herein as the inactive portion of the syringe reservoir 702. The sterile plunger syringe 700 is operated by expanding and contracting the volume of the active portion of the syringe reservoir 702 by moving the syringe plunger 703. Expansion and contraction of the volume of the active portion provides the necessary pressure imbalance to intake and expel fluids into the syringe reservoir 702.

The sterile plunger syringe 700 further includes a syringe plunger sealing device 709 secured to the syringe barrel 701 and configured to provide a syringe plunger seal 712. As used herein a “seal” as it relates to the syringe plunger sealing device refers to a fluid-tight, suitably liquid tight and in some embodiments gas-tight, junction, connection or fitting that does not allow the transfer of any substantive amount of fluid (including liquid and/or gas), suitably on the order of less than 1% of a volume of the reservoir 702, more suitably less than 0.1%, less than 0.01% and even more suitably less than 0.001% of a volume of the reservoir 702 is allowed to pass through the seal. The syringe plunger sealing device 709 is an example of a sterile sealing apparatus configured to maintain sterility of the syringe reservoir 702. The syringe plunger sealing device 709 surrounds the proximal end of the sterile plunger syringe 700 and the syringe plunger 703. The syringe plunger sealing device 709 may be a tube, bag, balloon, sock-like structure, accordion fold structure, or any other structure sufficient to enclose the proximal end of the sterile plunger syringe 700 and the syringe plunger 703. The syringe plunger sealing device 709 may be constructed from any suitable material, including polymers, plastics, etc.

A distal portion of the syringe plunger sealing device 709 may be secured to a proximal portion of the syringe barrel 701 distal of the syringe barrel flange 707. The syringe plunger sealing device 709 may also be secured to the syringe barrel flange 707 itself. Securement or attachment of the syringe plunger sealing device 709 to the syringe barrel 701 may be achieved through an adhesive, a heat bonding, a chemical bonding, or a sonic welding, by mechanical means such as clamping, and/or by any other suitable means. Mechanical means may removably secure the syringe plunger sealing device 709.

A proximal portion of the syringe plunger sealing device 709 may optionally be secured or attached to the syringe plunger rod flange 708. The syringe plunger sealing device 709 may encompass or surround the entire syringe plunger 703. In such embodiments, the syringe plunger rod flange 708 may be connected to the syringe plunger sealing device 709 such that movement of the syringe plunger 703 causes corresponding movement of the syringe plunger sealing device 709.

In further embodiments, the syringe plunger sealing device 709 has an opening in a proximal end. In such embodiments, the opening in the proximal end of the syringe plunger sealing device 709 is sealed to the syringe plunger rod flange 708 to maintain closure. An opening in the proximal end may be sealed to the syringe plunger rod flange 708 at a syringe plunger rod flange seal (not pictured).

In embodiments, the syringe plunger seal 712 is substantially fluid-tight. In embodiments, the syringe plunger seal 712 is substantially gas-tight for the purposes of maintaining sterility. In embodiments, the syringe plunger seal 712 is substantially liquid-tight but is not substantially gas-tight for example, to permit of gas vapor sterilization. In embodiments, the syringe plunger rod flange seal is substantially fluid-tight. In embodiments, the syringe plunger rod flange seal is substantially gas-tight. In embodiments, the syringe plunger rod flange seal is substantially liquid-tight but is not substantially gas-tight. Suitably on the order of less than 1% of a volume of the reservoir 702, more suitably less than 0.1%, less than 0.01% and even more suitably less than 0.001% of a volume of the reservoir 702, is allowed to pass through the seal.

The syringe plunger sealing device 709 operates to maintain or preserve the sterility of the syringe reservoir 702. Thus, the syringe plunger sealing device 709 maintains or preserves the sterility of the syringe reservoir 702 at the same level as the initial sterility of the packaged sterile plunger syringe 700 and prevents any degradation of sterility that may be caused by operation of the sterile plunger syringe 700 in an environment that is not sterile.

A conventional syringe operates by expansion and contraction of the active portion of the syringe reservoir. At the same time the active portion is expanded and contracted, the inactive portion of the syringe reservoir is respectively contracted and expanded. When the reservoir face is withdrawn from a distal end to a proximal end of the syringe barrel, the interior walls of the syringe barrel that were previously exposed to the environment outside of the syringe become the walls that define the active portion of the syringe reservoir. Accordingly, it is possible for contaminants from the environment outside of the syringe to cling to the walls of the syringe barrel and be introduced into the active portion of the syringe reservoir. It is also possible for contaminants from the inside of the syringe to similarly cling to the wall of the syringe barrel and therefore escape into the environment. This may be more likely when the syringe plunger is pushed into and out of the syringe barrel repetitively.

The syringe plunger sealing device 709 isolates the inactive portion of the syringe reservoir 702 during operation of the sterile plunger syringe 700. By isolating the inactive portion of the syringe reservoir 702 from the surrounding environment, contamination of the syringe reservoir 702 from the environment in which the sterile plunger syringe 700 is used can be prevented. The sterile plunger syringe 700 maintains sterility throughout the syringe reservoir 702, in both the active portion and the inactive portion, while the sterile plunger syringe 700 is operated. Sterility maintenance may require that the interconnect 710 be connected to a sterile or aseptic environment.

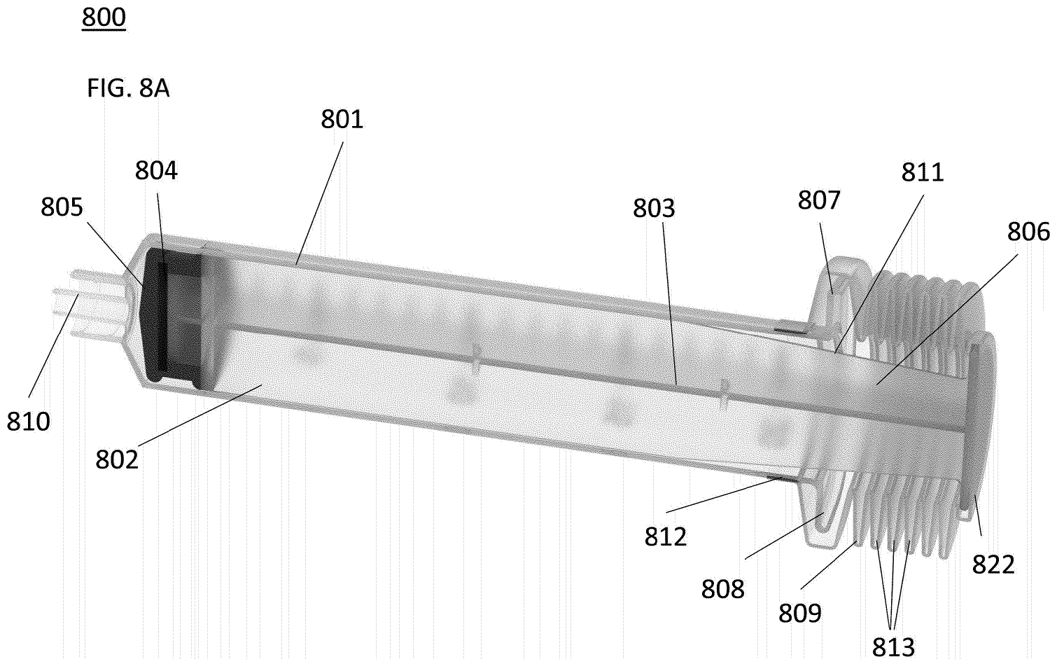

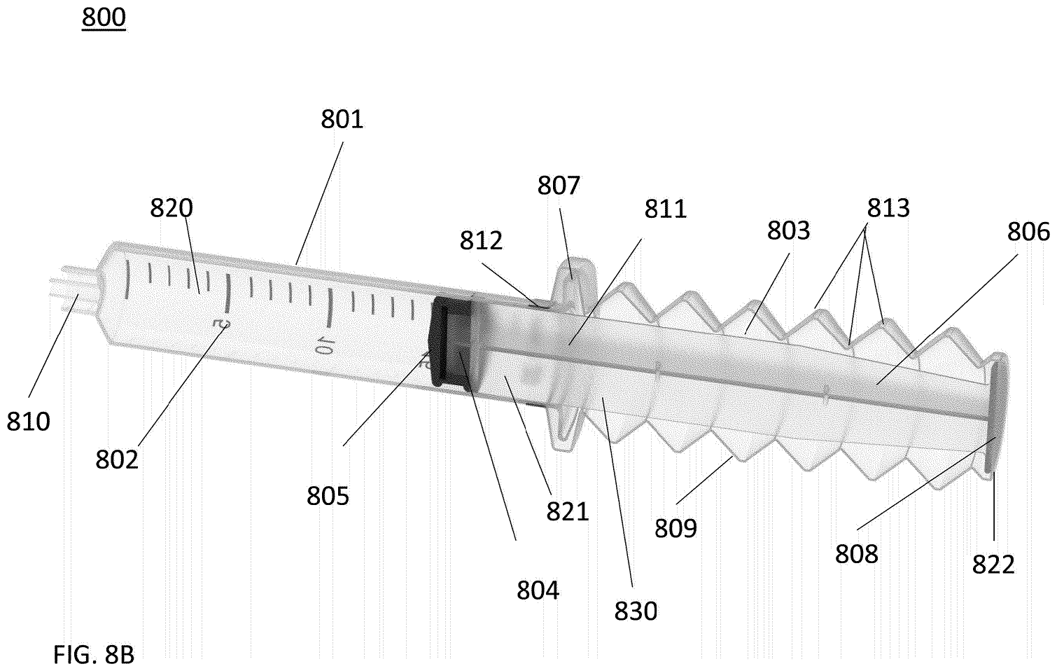

FIGS. 8A-8B illustrate a further sterile sampling device consistent with embodiments hereof. The sterile sampling device of FIGS. 8A-8B is a sterile plunger syringe 800 and may include a syringe barrel 801, a syringe plunger 803, a gasket 805, and a syringe plunger sealing device 809.

The syringe barrel 801 is generally similar to and includes all of the same features as the syringe barrel 701. The syringe barrel 801 defines a syringe reservoir 802 and includes an interconnect 810, a proximal opening 811 surrounded by a syringe barrel flange 807 The syringe barrel 801 is an example of a sample chamber defining a sample reservoir and the syringe reservoir is an example of a sample reservoir.

The syringe plunger 803 is generally similar to and includes all of the same features as the syringe plunger 703, including a syringe plunger rod 806, a syringe plunger rod flange 808 located at a proximal end of the syringe plunger rod 806, and a reservoir face 804 located at a distal end of the syringe plunger rod 806. The syringe plunger 803 is an example of a filling device configured to fill and empty the syringe reservoir 802. A gasket 805 is disposed over the reservoir face 804 and is generally similar to the gasket 705. A portion of the syringe reservoir 802 distal of the reservoir face 804 is referred to herein as the active portion 820 (shown in FIG. 8B) of the syringe reservoir 802 while a portion of the syringe reservoir 802 proximal of the reservoir face 804 is referred to herein as the inactive portion 821 (shown in FIG. 8B) of the syringe reservoir 802. The sterile plunger syringe 800 is operated by expanding and contracting the volume of the active portion 820 of the syringe reservoir 802 by moving the syringe plunger 803. Expansion and contraction of the volume of the active portion 820 provides the necessary pressure imbalance to intake and expel fluids into the syringe reservoir 802.

The sterile plunger syringe 800 further includes a syringe plunger sealing device 809 secured to the syringe barrel 801 and configured to provide a syringe plunger seal 812. The syringe plunger sealing device 809 is an example of a sterile sealing apparatus configured to maintain sterility of the syringe reservoir 802. The syringe plunger sealing device 809 surrounds the proximal end of the sterile plunger syringe 800 and the syringe plunger 803.

The syringe plunger sealing device 809 includes a plurality of accordion folds 813. The accordion folds 813 are configured to stack up, as shown in FIG. 8A, when the syringe plunger 803 is advanced into the syringe reservoir 802 and to stretch out when syringe plunger 803 is withdrawn towards a proximal end of the syringe reservoir 802, as shown in FIG. 8B.

In embodiments, the syringe plunger sealing device 809 may be configured with an opening at both the proximal and distal ends. In such embodiments, the syringe plunger sealing device 809 is configured as a tube pleated with accordion folds. The distal end of the syringe plunger sealing device 809 is sealed to the syringe barrel 801 at the syringe plunger seal 812 at a proximal portion of the syringe barrel 801. The syringe plunger seal 812 may be located on the syringe barrel flange 807 or may be located distal of the syringe barrel flange 807, on the cylindrical portion of the syringe barrel 801. The proximal end of the syringe plunger sealing device 809 is sealed to the syringe plunger 803 at the syringe plunger rod flange seal 822. Securement or attachment of the syringe plunger sealing device 809 to the syringe barrel 801 and the syringe plunger rod flange 808 may be achieved through an adhesive, a heat bonding, a chemical bonding, or a sonic welding, by mechanical means such as clamping, and/or by any other suitable means. Mechanical means may removably secure the syringe plunger sealing device 809.

In embodiments, the syringe plunger sealing device 809 may be configured with a single opening at the distal end at the location of the syringe plunger seal 812. The closed proximal end of the syringe plunger sealing device 809 may wrap over the plunger rod flange 808 and may optionally be sealed to the plunger rod flange 808 at a syringe plunger rod flange seal 822. Securement or attachment of the syringe plunger sealing device 809 to the syringe barrel 801 and the syringe plunger rod flange 808 may be achieved through an adhesive, a heat bonding, a chemical bonding, or a sonic welding, by mechanical means such as clamping, and/or by any other suitable means. Mechanical means may removably secure the syringe plunger sealing device 809.

As discussed above with respect to the syringe plunger seal 712, the syringe plunger seal 812 and the syringe plunger rod flange seal 822 may be substantially fluid-tight, substantially gas-tight, or substantially liquid-tight but not substantially gas-tight.

The syringe plunger sealing device 809 operates to maintain or preserve the sterility of the syringe reservoir 802 when the sterile plunger syringe 800 is operated. Sterility maintenance may require that the interconnect 810 be connected to a sterile or aseptic environment.

In embodiments, the syringe plunger sealing device 809 is configured to enclose a substantially similar volume throughout a range of operation by the syringe plunger 803. As used herein, a substantially similar volume refers to a volume that varies by less than 10%, less than 5%, or less than 1%. When the syringe plunger 803 is advanced into the syringe reservoir 802 (as shown in FIG. 8A), the syringe plunger sealing device 809 encloses an interior volume 830 (as shown in FIG. 8B), part of which is formed by the inactive portion 821 of the syringe reservoir 802 and part of which is formed by spaces and volumes between the syringe plunger rod 806 and the syringe plunger sealing device 809. When the syringe plunger rod 806 is withdrawn proximally through the syringe reservoir 802 (as shown in FIG. 8B), the part of the interior volume 830 formed from the inactive portion 821 decreases in size while the parts formed by the spaces and volumes between the syringe plunger rod 806 and the syringe plunger sealing device 809 increase. Throughout the range of operation of the syringe plunger rod 806, the interior volume 830 is maintained at substantially the same volume. As used herein, substantially the same volume means that any change in the volume of the interior volume 830 is not significant enough to interfere with operation of the sterile plunger syringe 800. For example, substantially the same volume may include volume changes of less than 5%, 4%, 3%, 2%, and/or 1%. If the volume of the interior volume 830 differs during operation, it could create pressure imbalances that interfere with operation of the sterile plunger syringe 800. Reducing the interior volume 830 is resisted by the pressure inside the interior volume 830 while expanding the interior volume 830 is resisted by the pressure outside of the interior volume 830.

The interior volume 830 may be maintained at substantially the same volume through the range of operation by various design aspects of the syringe plunger sealing device 809. In embodiments, the accordion folds 813 are sized and configured such that the interior volume 830 remains substantially the same size as they are folded and/or stretched. In further embodiments, the syringe plunger sealing device 809 is flexible so as to permit the interior volume 830 to remain at substantially the same volume during operation through flexing of the syringe plunger sealing device 809. In further embodiments, both methods may be employed, with both the accordion folds 813 and flexibility providing a portion of the maintenance of the interior volume 830.

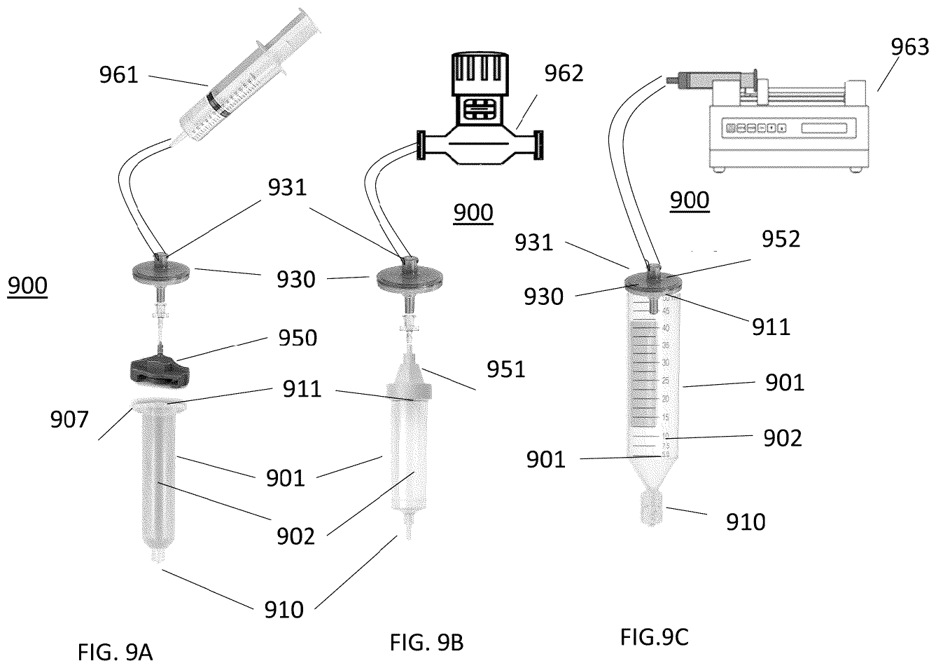

FIGS. 9A-9C illustrate sterile sampling devices consistent with embodiments hereof

FIGS. 9A-9C illustrate embodiments of a sterile sampling device 900. The sterile sampling device 900 includes a sample container 901, a filter 930, and a filter interconnect 931. FIGS. 9A-9C illustrate sterile sampling devices 900 employing differing container-filter connectors 950/951/952 and differing filling devices 961/962/963.

The sample container 901 defines a sample reservoir 902 and includes an interconnect 910 and a proximal opening 911. The sample container 901 is an example of a sample chamber defining a sample reservoir. The sample container 901 may be a syringe reservoir or any other suitable container. The sample container 901 may be a syringe or other cylindrical container and may be constructed of any suitable material, including for example, polyethylene, polycarbonate, polypropylene, stainless steel, etc. In embodiments, the sample container 901 may include a flange 907, such as a syringe barrel flange. In embodiments, the sample container 901 may include graduated volume markings.

The container filter connector 950/951/952 connects to the sample container 901 at the proximal opening 911. The container filter connector 950/951/952 provides a substantially fluid-tight seal, substantially gas-tight seal, or a seal that is substantially liquid tight but not gas-tight. The container filter connector 950/951/952 may be any suitable connector. For example, the container-filter connector 950, as illustrated in FIG. 9A, is a syringe barrel adapter configured to latch to the flange 907 and seal the proximal opening 911. In further embodiments, as illustrated in FIG. 9B, the container filter connector 951 is a bayonet fitting that screws onto the proximal opening 911 of the sample container 901. In still further embodiments, as illustrated in FIG. 9C, the container-filter connector 952 is integral with the filter 930 and the filter interconnect 931.

The container filter connector 950/951/952 connects to the filter 930. In embodiments, the filter 930 a 0.22 micrometer hydrophobic filter Other suitable filters may include 0.2 micron hydrophobic filter or any filter having a membrane hydrophobicity dissimilar to the fluid being contained within the sampling device where the pore size of the membrane is small enough to contain the fluid but large enough for air/gas to pass through the membrane such that the performance of the sterile sampling device is not substantially impacted. The filter 930 is an example of a sterile sealing apparatus configured to maintain sterility of the sample reservoir 902. A filter interconnect 931 is disposed on a proximal side of the filter 930.

Connected to the filter interconnect 931 is a filling device 961/962/963 configured to fill and empty the sample reservoir 902. The filling device 961/962/963 may be another syringe, a pump, a syringe pump, a vacuum, or any other device suitable for causing a sample to be drawn into the syringe reservoir through the interconnect 910. For example, FIG. 9A illustrates use of a syringe filling device 961, FIG. 9B illustrates use of a pump filling device 962, and FIG. 9C illustrates use of a syringe pump filling device 963.

When operated, the sterile sampling device 900 maintains sterility of the sample reservoir 902. Sterility maintenance may require that the interconnect 910 be connected to a sterile or aseptic environment. The filling device 961/962/963 is operated to reduce the pressure in the sample reservoir 902, causing the intake of a sample or other fluid. To expel the sample or other fluid, the filling device 961/962/963 is operated to increase the pressure in the sample reservoir 902. The filter 930 permits enough gas (e.g., air) to pass through to provide appropriate pressure reductions and increases to cause intake and expulsion of the sample or fluid. The filter 930, however, does not permit the passage of contaminants. Thus, the filter 930 operates to maintain the sterility of the sample reservoir 902 during operation of the sterile sampling device 900. Sterility maintenance of the sample reservoir 902 does not require that the filling device be similarly sterilized.

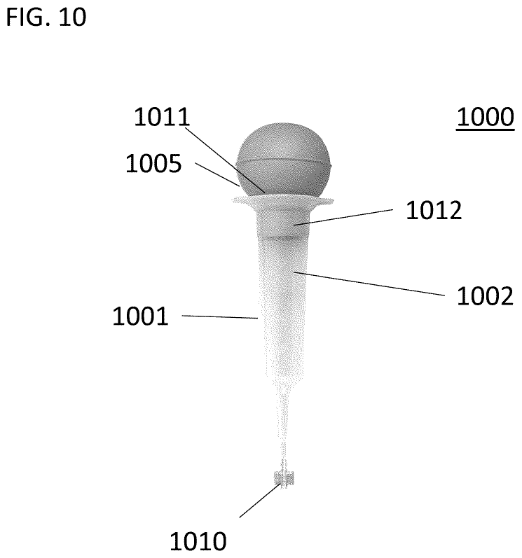

FIG. 10 illustrates a further sterile sampling device consistent with embodiments hereof. The sterile plunger syringe 1000 is a sterile sampling device. The sterile plunger syringe 1000 includes a syringe barrel 1001 defining a syringe reservoir 1002 and having an interconnect 1010 located at a distal end. The syringe barrel 1001 is an example of a sample chamber and the syringe reservoir is an example of a sample reservoir.

The sterile plunger syringe 1000 further includes a sampling bulb 1005 sealed to a proximal opening 1011 the syringe reservoir 1002 by sterile seal 1012. The sterile seal 1012 is substantially fluid-tight and, in some embodiments, substantially gas-tight. In some embodiments, the sterile seal 1012 is substantially liquid-tight but not substantially gas-tight. The sampling bulb 1005 is operated to intake and expel fluids from the syringe reservoir 1002. The sampling bulb 1005 is thus another example of a filling device configured to fill and empty the syringe reservoir 1002. The sampling bulb 1005 in conjunction with the sterile seal 1012 is configured to prevent any contaminants from the environment from entering the syringe reservoir 1002. Thus, the sampling bulb 1005 and sterile seal 1012 are another example of a sterile sealing apparatus configured to maintain the sterility of the syringe reservoir 1002.

In operation, the sampling bulb 1005 is squeezed or compressed to expel air (or other gas) from the syringe reservoir 1002. When the sampling bulb 1005 is released, it returns to its original shape, drawing fluid into the syringe reservoir 1002 through the interconnect 1010. Because the sampling bulb 1005 and syringe reservoir 1002 are a closed system, no contaminants can enter the syringe reservoir 1002 from the environment.

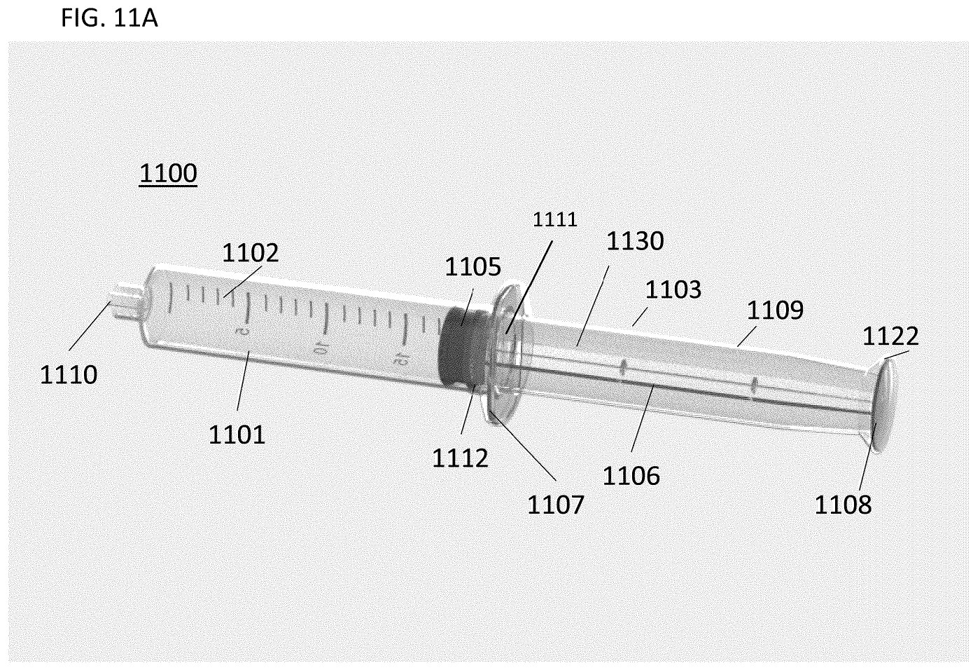

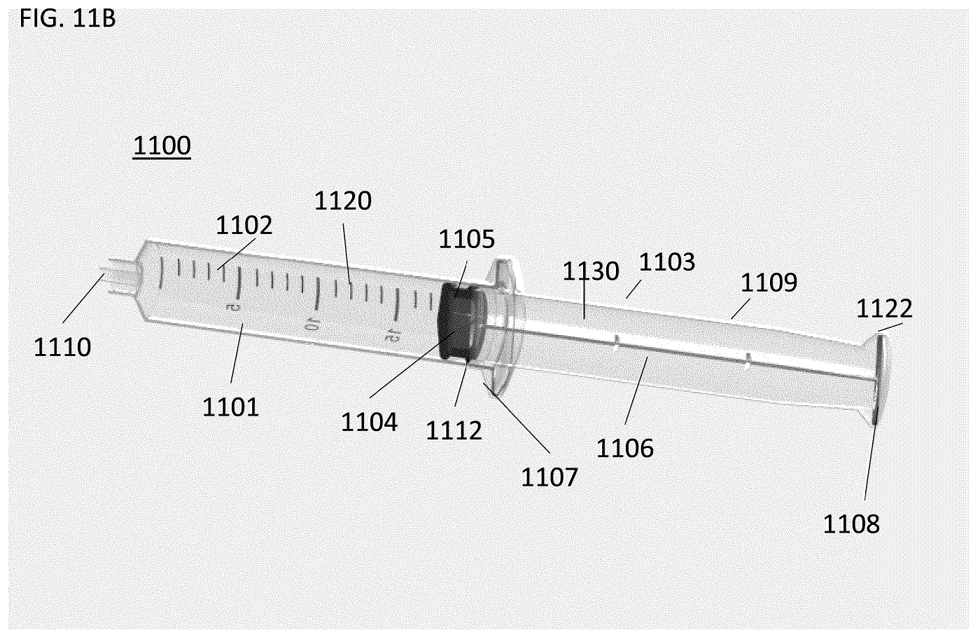

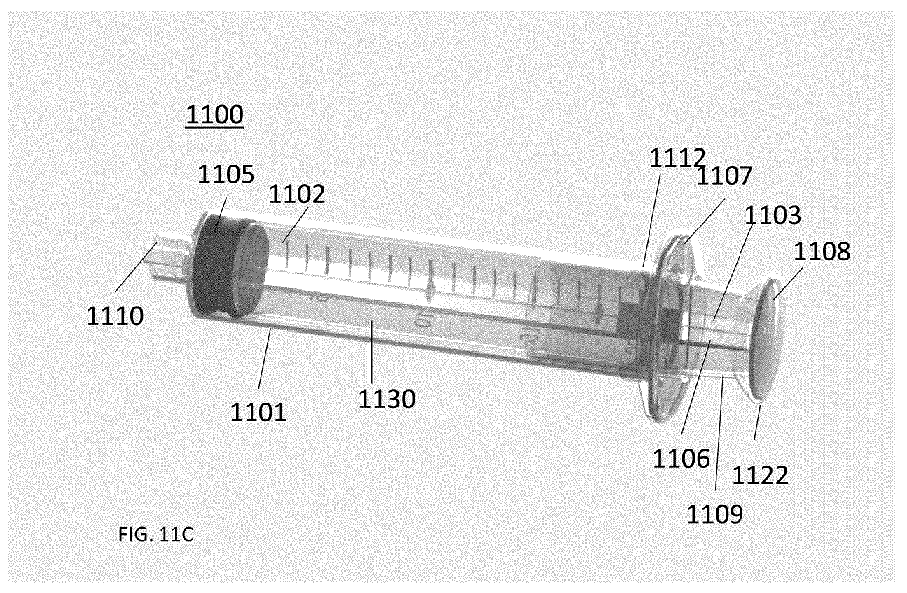

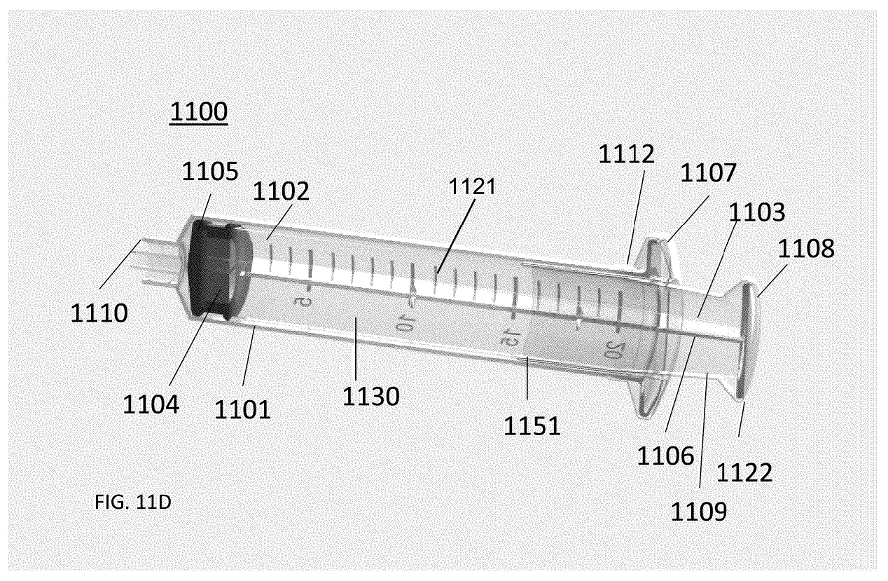

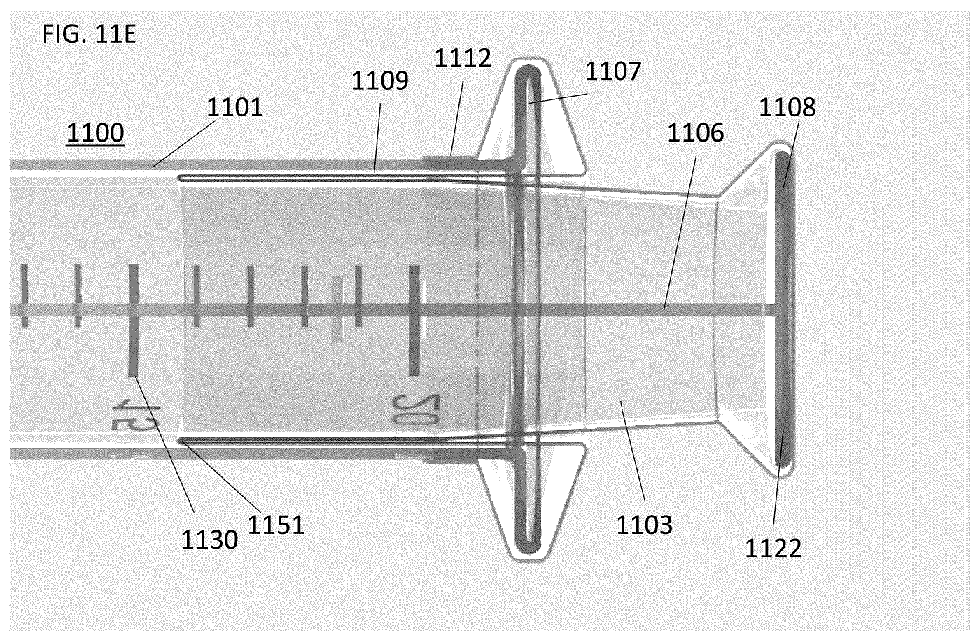

FIGS. 11A-11E illustrate a further sterile sampling device consistent with embodiments hereof. The sterile sampling device of FIGS. 11A-11E is a sterile plunger syringe 1100 and may include a syringe barrel 1101, a syringe plunger 1103, a gasket 1105, and a syringe plunger sealing device 1109. FIGS. 11A and 11B are perspective and cut-away views of the sterile plunger syringe 1100 with the syringe plunger 1103 inserted fully into the syringe barrel 1101. FIGS. 11C and 11D are perspective and cut-away views of the sterile plunger syringe 1100 with the syringe plunger 1103 fully withdrawn but still seated within the syringe barrel 1101. FIG. 11E is a closeup view of the syringe plunger sealing device 1109.

The syringe barrel 1101 is generally similar to and includes all of the same features as the syringe barrel 701 or syringe barrel 801. The syringe barrel 1101 defines a syringe reservoir 1102 and includes an interconnect 1110, a proximal opening 1111 surrounded by a syringe barrel flange 1107 The syringe barrel 1101 is an example of a sample chamber defining a sample reservoir and the syringe reservoir is an example of a sample reservoir.

The syringe plunger 1103 is generally similar to and includes all of the same features as the syringe plunger 703 and the syringe plunger 803, including a syringe plunger rod 1106, a syringe plunger rod flange 1108 located at a proximal end of the syringe plunger rod 1106, and a reservoir face 1104 located at a distal end of the syringe plunger rod 1106. The syringe plunger 1103 is an example of a filling device configured to fill and empty the syringe reservoir 1102. A gasket 1105 is disposed over the reservoir face 1104 and is generally similar to the gasket 705 and the gasket 805. A portion of the syringe reservoir 1102 distal of the reservoir face 1104 is referred to herein as the active portion 1120 (shown in FIG. 11B) of the syringe reservoir 1102 while a portion of the syringe reservoir 1102 proximal of the reservoir face 1104 is referred to herein as the inactive portion 1121 (shown in FIG. 11D) of the syringe reservoir 1102. The sterile plunger syringe 1100 is operated by expanding and contracting the volume of the active portion 1120 of the syringe reservoir 1102 by moving the syringe plunger 1103. Expansion and contraction of the volume of the active portion 1120 provides the necessary pressure imbalance to intake and expel fluids into the syringe reservoir 1102.

The sterile plunger syringe 1100 further includes a syringe plunger sealing device 1109 secured to the syringe barrel 1101 and configured to provide a syringe plunger seal 1112. The syringe plunger sealing device 1109 is an example of a sterile sealing apparatus configured to maintain sterility of the syringe reservoir 1102. The syringe plunger sealing device 1109 surrounds the proximal end of the sterile plunger syringe 1100 and the syringe plunger 1103.

The syringe plunger sealing device 1109 is a flexible tube or bag sealed to the syringe barrel 1101 at one end and sealed to the syringe plunger rod flange at a second end. Materials for constructing syringe plunger sealing device 1109 include various plastics and polymers, and suitably include plastic tubes or bags that have a thickness on the order of about 100 mm to about 1-2 mm, for example between about 100 mm to about 1 mm, or about 100 mm to about 800 mm.

The syringe plunger sealing device 1109 is sealed to the syringe barrel 1101 at the syringe plunger seal 1112 at a proximal portion of the syringe barrel 1101. The syringe plunger seal 1112 may be located on the syringe barrel flange 1107 or may be located distal of the syringe barrel flange 1107, on the cylindrical portion of the syringe barrel 1101. The proximal end of the syringe plunger sealing device 1109 is sealed to the syringe plunger 1103 at the syringe plunger rod flange seal 1122. Securement or attachment of the syringe plunger sealing device 1109 to the syringe barrel 1101 and the syringe plunger rod flange 1108 may be achieved through an adhesive, a heat bonding, a chemical bonding, or a sonic welding, by mechanical means such as clamping, and/or by any other suitable means. Mechanical means may removably secure the syringe plunger sealing device 1109.

In embodiments, the syringe plunger sealing device 1109 may be configured with an opening at both the proximal and distal ends. In such embodiments, the syringe plunger sealing device 1109 is configured as a tube. In such an embodiment, the ends of the syringe plunger sealing device 1109 are closed through the syringe plunger seal 1112 and the syringe plunger rod flange seal 1122.

In embodiments, the syringe plunger sealing device 1109 may be configured with a single opening at the distal end at the location of the syringe plunger seal 1112. The closed proximal end of the syringe plunger sealing device 1109 may wrap over the plunger rod flange 1108 and may optionally be sealed to the plunger rod flange 1108 at a syringe plunger rod flange seal 1122. Securement or attachment of the syringe plunger sealing device 1109 to the syringe barrel 1101 and the syringe plunger rod flange 1108 may be achieved through an adhesive, a heat bonding, a chemical bonding, or a sonic welding, by mechanical means such as clamping, and/or by any other suitable means. Mechanical means may removably secure the syringe plunger sealing device 1109.

As discussed above with respect to the syringe plunger seal 712, the syringe plunger seal 1112 and the syringe plunger rod flange seal 1122 may be substantially fluid-tight, substantially gas-tight, or substantially liquid-tight but not substantially gas-tight.

The syringe plunger sealing device 1109 operates to maintain or preserve the sterility of the syringe reservoir 1102 when the sterile plunger syringe 1100 is operated. Sterility maintenance may require that the interconnect 1110 be connected to a sterile or aseptic environment.

In embodiments, the syringe plunger sealing device 1109 is configured to enclose a substantially similar volume throughout a range of operation by the syringe plunger 1103. The syringe plunger sealing device 1109 encloses an interior volume 1130, the shape of which changes dynamically with operation of the sterile plunger syringe 1100. The syringe plunger sealing device 1109 is configured such that, when the syringe plunger 1103 is withdrawn but still seated in the syringe barrel 1101 (as shown in FIGS. 11A and 11B), the syringe plunger sealing device 1109 has an inner diameter similar to the inner diameter of the syringe barrel 1101. In this configuration, the interior volume 1130 is defined by the volume between the syringe plunger sealing device 1109 and the syringe plunger rod 1106 plus any additional volume between the syringe plunger rod and the distal end of the syringe barrel 1101.

When the syringe plunger 1103 is pushed into the syringe barrel 1101, the excess material of the syringe plunger sealing device 1109, which is flexible, is pushed into the syringe barrel 1101. The flexible syringe plunger sealing device 1109 folds over itself at fold 1151. FIG. 11E illustrates the fold 1151 and the syringe plunger sealing device 1109 pushed partially into the syringe barrel 1101. The material of the syringe plunger sealing device 1109 is thin and clings to the wall of the syringe barrel 1101. Accordingly, the syringe plunger sealing device 1109 portion that is within the syringe barrel 1101 occupies only a negligible portion of the total volume inside the syringe barrel 1101. As used herein, a negligible portion refers to a volume portion that is less than 5%, less than 2%, less than 1%, and/or less than 0.5% of the volume inside the syringe barrel 1101.

When the syringe plunger 1103 is pushed partially into the syringe barrel 1101, the interior volume 1130 is defined by the volume between the syringe plunger sealing device 1109 and portion of the syringe plunger rod 1106 protruding from the syringe barrel 1101 and by the volume between the syringe barrel 1101 and the portion of the syringe plunger rod 1106 disposed within the syringe barrel 1101 (minus the negligible portion occupied by the folded syringe plunger sealing device 1109). When the syringe plunger 1103 is pushed fully into the syringe barrel 1101, the interior volume 1130 is defined by the volume between the syringe barrel 1101 and the portion of the syringe plunger rod 1106 disposed within the syringe barrel 1101 (minus the negligible portion occupied by the folded syringe plunger sealing device 1109) and the remaining volume between the syringe plunger sealing device 1109 and the portion of the syringe plunger rod 1106 still protruding from the syringe barrel 1101.

In each of the above described configurations, partial insertion, full insertion, and full withdrawal, as well as all configurations in between, the interior volume 1130 remains substantially the same because the shape of the interior volume 1130 varies only negligibly. The length of the interior volume 1130 does not change, as it is represented by the distance between the back of the reservoir face 1104 and the syringe plunger rod flange 1108. The diameter of the interior volume 1130 changes only negligibly because, in the various configurations, the diameter of the interior volume is defined partially by the syringe barrel 1101 (minus any portion of the syringe plunger sealing device 1109 inside the syringe barrel 1101) and partially by the syringe plunger sealing device 1109, which, as discussed above is similar to the diameter of the syringe barrel 1101. As the syringe plunger 1103 moves in and out of the syringe barrel 1101, the relative proportion of the interior volume 1130 that is inside the syringe barrel 1101 changes, but the approximate diameter of the interior volume 1130 does not change. Accordingly, the interior volume 1130 remains substantially the same throughout any configuration of the sterile plunger syringe 1100.

In further embodiments, flexibility of the syringe plunger sealing device 1109 operates to balance the negligible volume change of the interior of the syringe barrel 1101 when the syringe plunger 1103 is pressed into the syringe barrel 1101. Any decrease in a volume of the interior volume 1130 that may be caused by the syringe plunger sealing device 1109 occupying a portion of the interior of the syringe barrel 1101 may be counteracted by a corresponding increase in a volume of the interior volume 1130 which may be caused by a slight expansion, e.g., through stretching or freedom of movement, of the portion of the syringe plunger sealing device 1109 that remains outside the syringe barrel 1101. Accordingly, the flexibility of the syringe plunger sealing device 1109 may serve to ensure that that the interior volume 1130 remains substantially the same throughout all configurations of the syringe plunger 1103.

As discussed above, the volume of the interior volume 1130 differs during operation, it could create pressure imbalances that interfere with operation of the sterile plunger syringe 1100. Reducing the interior volume 1130 is resisted by the pressure inside the interior volume 1130 while expanding the interior volume 1130 is resisted by the pressure outside of the interior volume 1130.

The various sterile sample devices described herein are suitably used in conjunction with a cassette 602 of an automated cell engineering system 600 as described herein. Such sample devices allow for the removal and/or introduction of required or desired biological material samples, cells, media, etc., without concern of compromising the sterile integrity of the systems, and without disturbing the cells or biological materials being produced therein.

In further embodiments, the various sterile sampling devices described herein may be operated or employed with any system that maintains a sterile interior while operating in an environment with that is not sterile. Sterility concerns discussed herein with respect to automated cell engineering systems may equally apply to further systems that include a functionally enclosed sterile interior. Such systems may benefit from operation in a non-sterile environment and thus may benefit from the use of the sterile sampling devices and methods disclosed herein.

In embodiments, any or all of the above-discussed sterile sampling devices may be operated manually or automatically. Manual operation includes manipulation of the sterile sampling device by an operator via their hands and/or through the use of one or more tools, fixtures, or devices configured to assist in the operation. Automatic operation includes the use of actuator driven systems or devices to operate sterile sampling devices as described herein. Appropriate actuators may include syringe pumps, motors, servomotors, vacuum pumps, etc. Any or all of the devices described above may be modified to accommodate automatic operation. For example, for use in a syringe pump, a sterile plunger syringe may include a mechanical portion configured for interconnect with the operative portions of the syringe pump. In further embodiments, automated sterile sampling devices may be provided. Automated sterile sampling devices may include cartridge- or cassette- based sampling devices including interconnects, a sample chamber and sample reservoir, appropriate fluidic passages, appropriate gas passages, appropriate valves, a filling device configured to draw fluids into and expel fluids from the sample chamber, and a sterile sealing apparatus configured to maintain sterility during operation of the automated sterile sampling device.

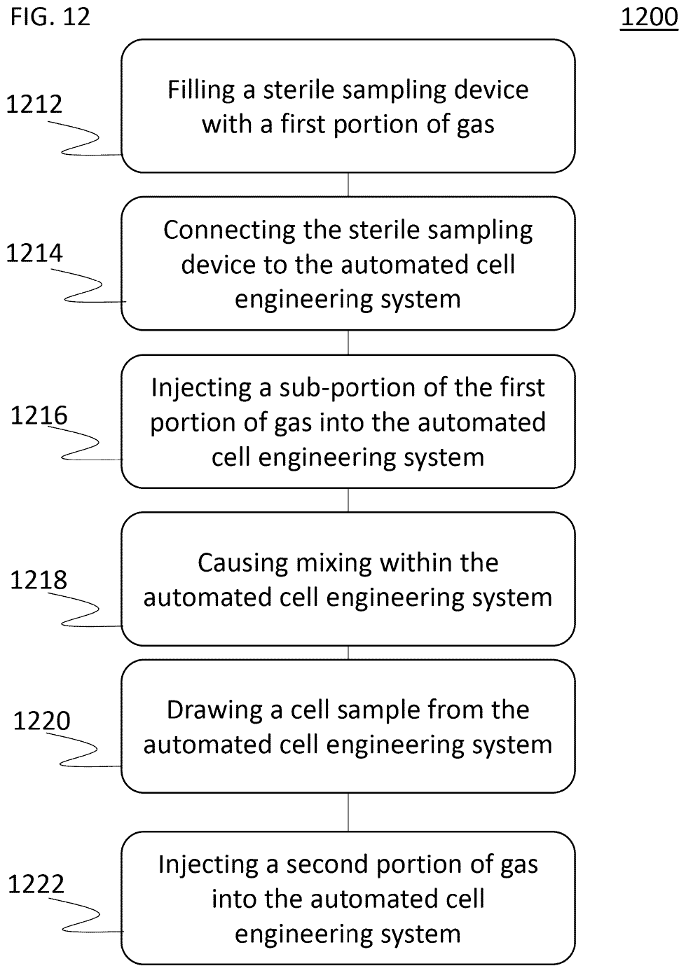

FIG. 12 is a flow chart illustrating steps in a sterile sampling method. The sterile sampling method 1200 of FIG. 12 may be carried out using any sterile sampling device disclosed herein, via manual or automated methods. The sterile sampling method 1200 does not require that all of the below-discussed operations be carried out in the order described. Some of the operations may be omitted, rearranged, and/or repeated multiple times without departing from the scope of the sterile sampling method as described. The sterile sampling method 1200 may be carried out to withdraw a sample from a cell engineering system, as described herein. The withdrawn sample may include, for example, a cell culture, a biological material sample, a reagent sample, or any other fluid or media that may be required to be withdrawn from the cell engineering system.

A sterile sampling device may be provided to carry out the sterile sampling method. A sterile sampling device suitable for carrying out the sterile sampling method 1200 may include at least a sample chamber defining a sample reservoir and having an interconnect, a filling device, and a sterility sealing apparatus, as described herein. Examples of these aspects of a sterile sampling device are described herein and are suitable for carrying out the sterile sampling method 1200 described below.

In an operation 1212, the sterile sampling method 1200 includes filling a sample reservoir of a sterile sampling device with a first gas portion. The first gas portion may be air and/or may be any other suitable gas. The first gas portion may be sterile and/or may be drawn from a sterile environment. In embodiments, the sterile sampling device may be packaged and provided such that it contains a sterile gas portion. In further embodiments, the filling device may be employed to fill the sample reservoir with the first gas portion.

In an operation 1214, the sterile sampling method 1200 includes connecting the sterile sampling device to the cell engineering system. The interconnect of the sterile sampling device is suitably connected to a port of cassette of the cell engineering system. Prior to connection, the port of the cell engineering system may be appropriately sterilized, e.g., via sterile wipes or spray, to prevent contamination during the sampling procedure. In embodiments, a sterile extension line may be used to connect the interconnect of the sterile sampling device to the port of the cell engineering system. Connections between the sterile sampling device, the extension line, and the cell engineering system port may be achieved via any suitable means, including Luer locks, closed Luer connectors, press fit connectors, snap fit connectors, etc.

In an operation 1216, the sterile sampling method 1200 includes injecting a sub-portion of the first gas portion, via the filling device, to clear a feed line within the cell engineering system. The sterile sampling device is operated to inject part or all of the first gas portion into the cell engineering system. The injected gas travels into a feed line of the cell engineering system and into a sample containing system culture reservoir of the cell engineering system. The feed line of the cell engineering system is a conduit for the sample that connects the system culture reservoir to the ports of the cell engineering system. Injecting gas through the feed line serves to push any sample that has collected in the feed line back into the system culture reservoir. This step may serve to homogenize the sample in the cell engineering system culture reservoir prior to sample withdrawal.

In an operation 1218, the sterile sampling method 1200 includes mixing, via operation of the filling device, the sample within the cell engineering system by the sterile sampling device. The sample in the cell engineering system culture reservoir may be further homogenized through a mixing procedure applied via the sterile sampling device. Such mixing may be achieved by withdrawing a mixing sample into the sterile sampling device and then injecting the mixing sample back into the cell engineering system culture reservoir. Mixing via withdrawal and reinjection may be conducted any suitable number of times.

In an operation 1220, the sterile sampling method 1200 includes drawing, via the filling device, a sample from the cell engineering system with the sterile sampling device. A specific volume of sample may be withdrawn. Withdrawing the sample may be conducted after the cell engineering system feed line is cleared and/or after a mixing procedure is conducted.

In an operation 1222, the sterile sampling method 1200 includes injecting, via the filling device, a second portion of gas into the cell engineering system. A second portion of gas is injected into the cell engineering system after withdrawal of the sample to clear the feed line. After sample withdrawal through the cell engineering system ports, a portion of sample may remain in the feed line. A second portion of gas may be injected from the sterile sampling device back into the cell engineering system to clear the feed line.

As discussed above, the sterile sampling method 1200 does not require that each of the operations 1212 to 1222 be carried out in the order described. Any suitable arrangement of any number of the operations 1212 to 1222 may be carried out in a sterile sampling method in various embodiments. For example, in an embodiment, a sterile sampling device may be connected to the automated cell engineering system in an operation 1214 and a sample may be withdrawn by the sterile sampling device in an operation 1222 without any intervening operations. In a further embodiment, a sample reservoir of a sterile sampling device may be filled with a first gas portion in an operation 1212 and then connected to the automated cell engineering system in an operation 1214. A sub-portion of the first gas portion may then be injected into the automated cell engineering system in an operation 1216 prior to withdrawal of a sample from the automated cell engineering system in an operation 1220. After the withdrawal, a second portion of gas may be injected into the automated cell engineering system in an operation 1222 to clear a feed line of the automated cell engineering system. In still further embodiments, injection of the second portion of gas may be omitted. Still further embodiments may include causing mixing in an operation 1218 while excluding one or both of the gas injection operations 1216 and 1222. In additional embodiments, all of the previously described embodiments may be carried out without filling the sterile sampling device with a first portion of gas at an operation 1212, either because gas injection steps are omitted or because the sterile sampling device is provided pre-filled with the first portion of gas.

Throughout the sterile sampling method 1200 and operation of the sterile sampling device, the sterile sealing apparatus is employed to maintain the sterility of the sample reservoir. Maintaining sterility of the sample reservoir entails maintaining sterility of any sample that is drawn into the sample reservoir and, therefore, maintaining the sterility of any sample contained within the cell engineering system. Through the operation of the sterile sealing apparatus, the sterile sampling method 1200 may be carried out in a non-sterile environment without compromising the sterility of the cell engineering system.

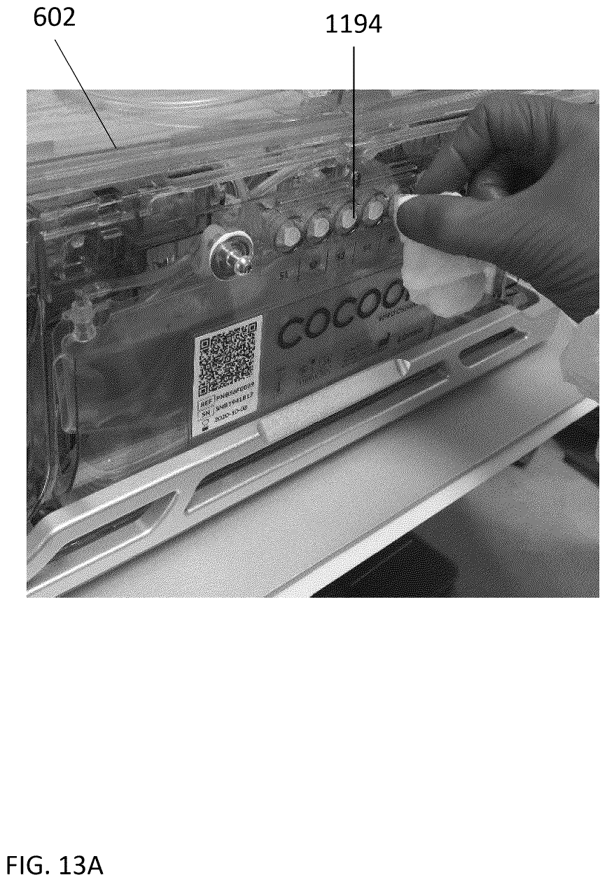

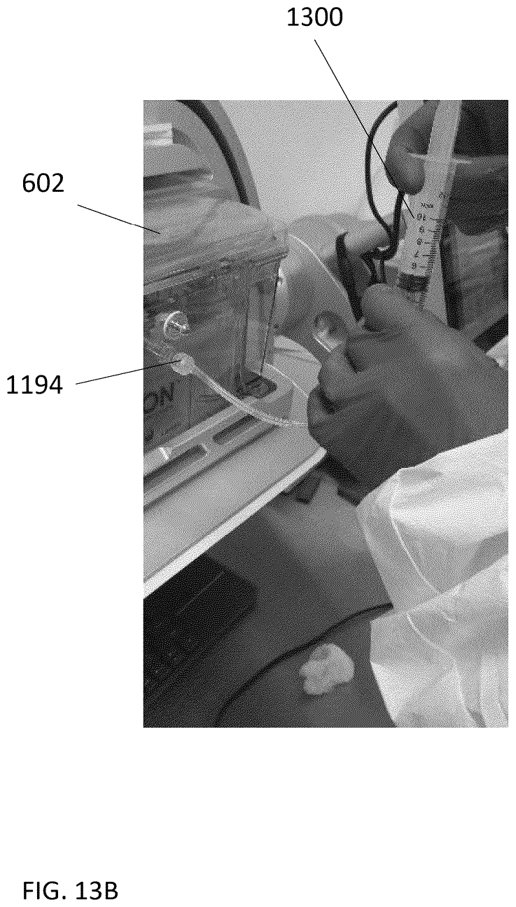

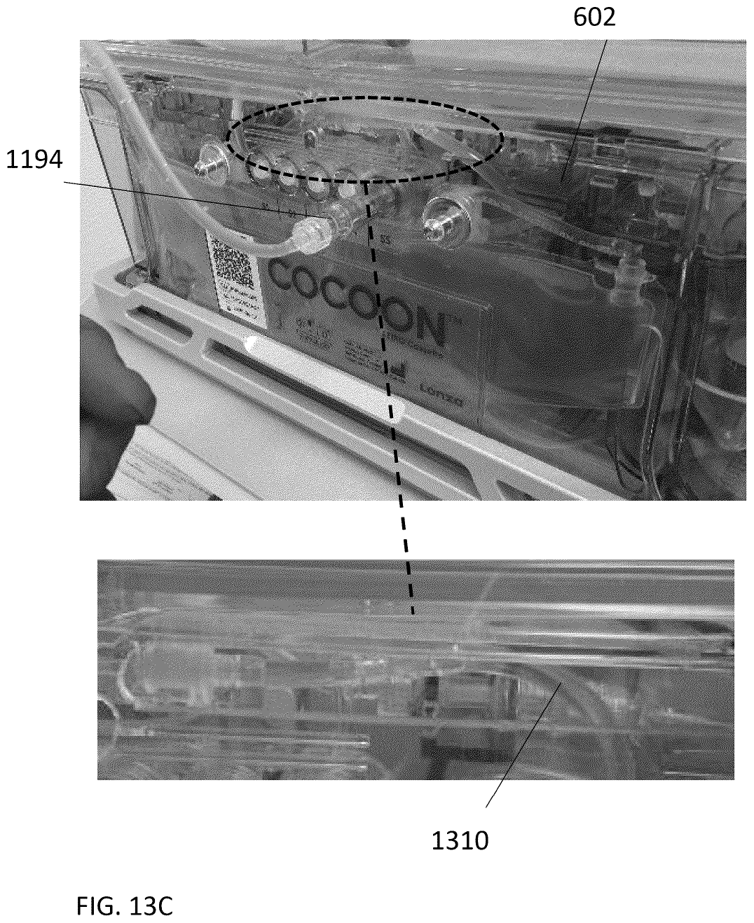

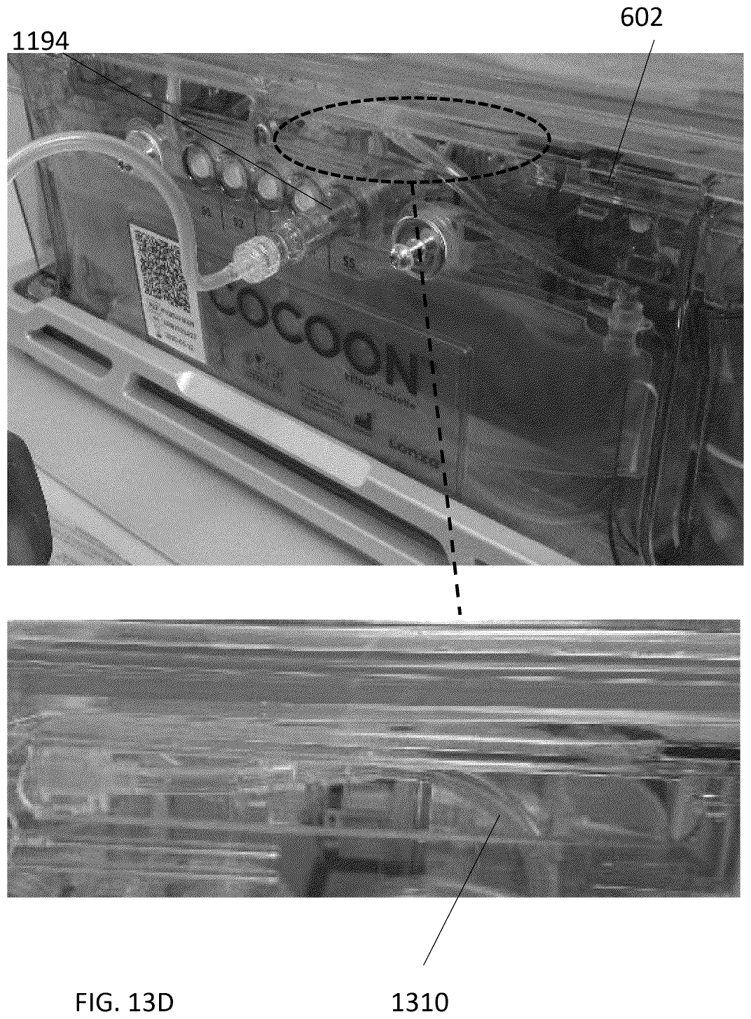

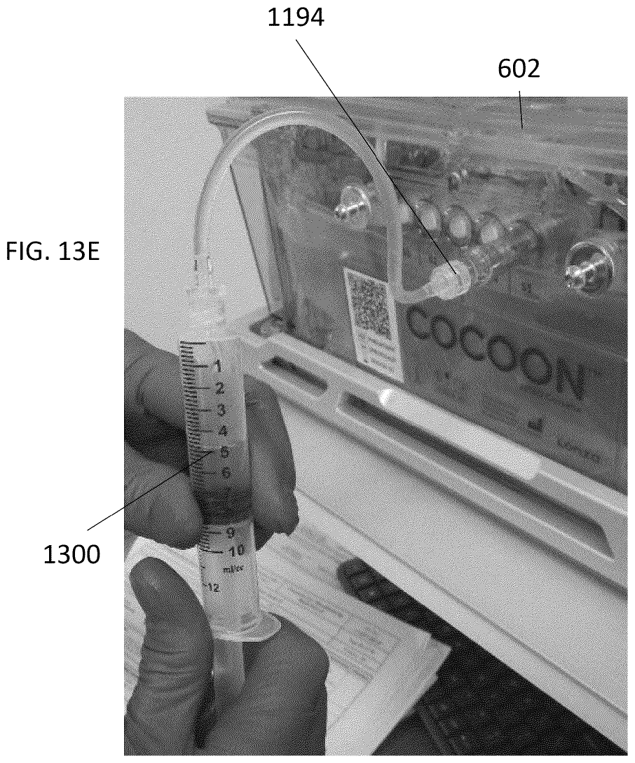

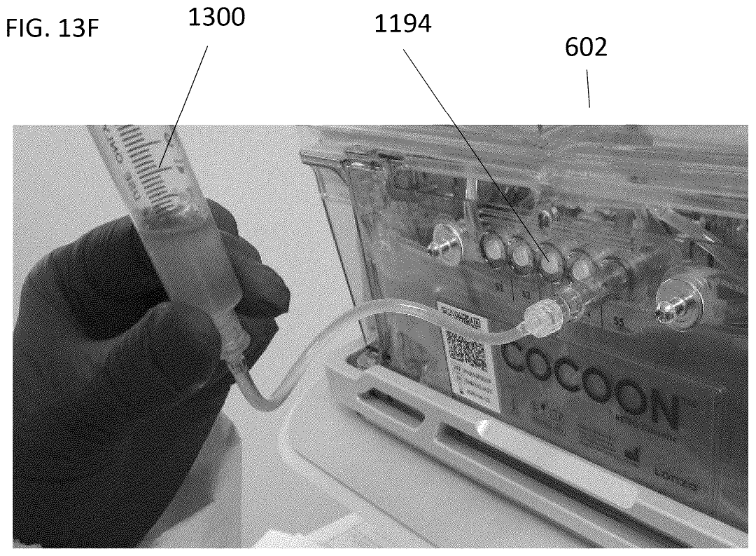

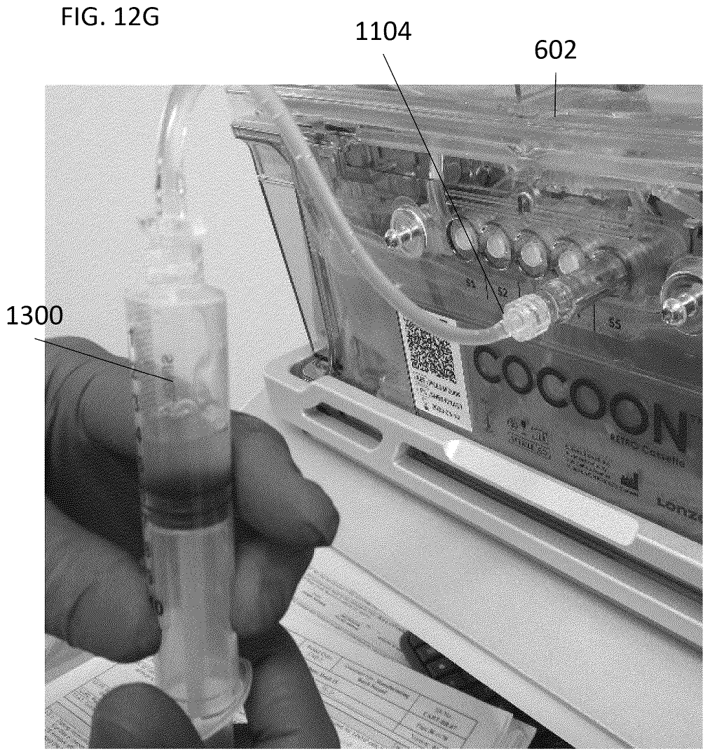

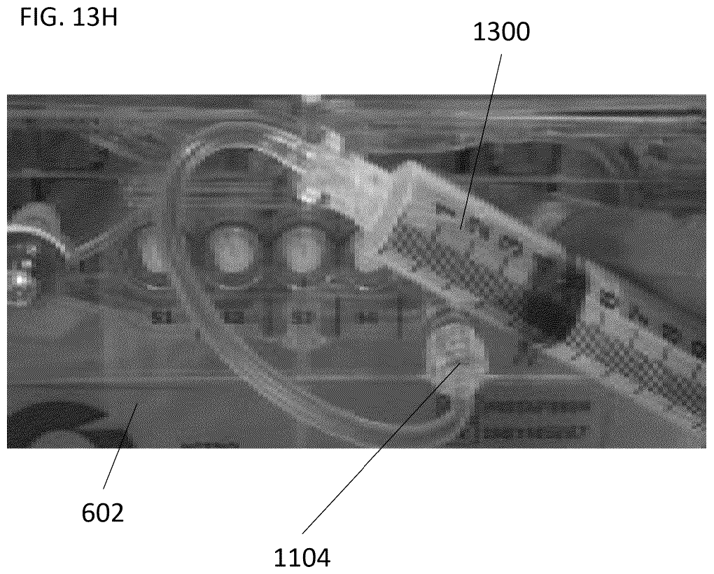

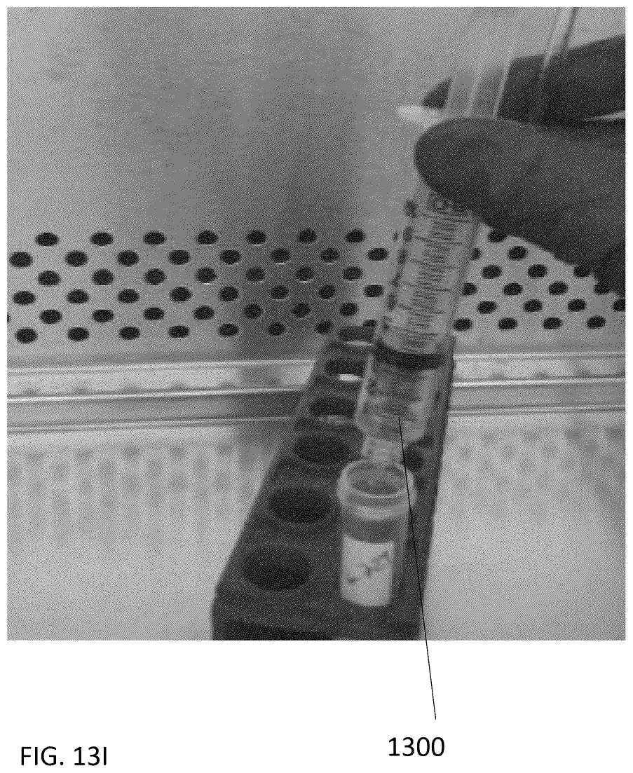

FIGS. 13A-13K illustrate a specific embodiment of the sterile sampling method described with respect to FIG. 12. The methods and procedures described with respect to FIGS. 13A-13K may also be carried out through suitable automated means. As illustrated, a sterile sampling device 1300 is employed in the sterile sampling procedure. The sterile sampling device 1300 may include any of the sterile sampling devices discussed herein and any other device capable of preforming the steps of the procedure described. The specific embodiment of FIGS. 13A-13K is representative of the sterile sampling method 1200 but does not limit it. As discussed above, the sterile sampling method 1200 is not limited to the devices or actions illustrated or discussed with respect to FIGS. 13A-13K.

FIG. 13A illustrates an initial step of providing exterior sterilization to ports 1194 of the cassette 602 of an automated cell engineering system 600. The sterile sampling devices discussed herein provide a sterile device for sampling in a non-sterile environment. Prior to connecting a sterile sampling device to the automated cell engineering system, the ports 1194 are sterilized to remove any contaminants from the non-sterile environment.

FIG. 13B illustrates a step of connecting a sterile sampling device 1300 to the ports 1094 of the cassette 602 of the automated cell engineering system 600 and an optional step of introducing or injecting a portion of gas into the automated cell engineering system. Prior to connecting the sterile sampling device 1300 to the ports 1094 a portion of gas is introduced into the sterile sampling device 1300. In embodiments, the gas may be air or any other suitable gas. In embodiments, the gas may be sterile, e.g., at least as sterile as the environment of the automated cell engineering system. The portion of gas may be injected from the sterile sampling device 1300 into the automated cell engineering system to clear a feed line. FIG. 13C illustrates the feed line 1310 containing fluid prior to air injection. The feed line 1310 contains fluid. FIG. 13D illustrates the feed line 1310 clear of fluid after air injection.

The feed line 1310 may be cleared via gas injection to allow appropriate mixing of the fluid to be sampled. For example, the fluid in the feed line 1310 may not be well mixed with the fluid in the system culture reservoir of the automated cell engineering system 600. This may occur, for example, due to additions (i.e., fluids added) or alterations (e.g., cell growth) of the fluid in the system culture reservoir. Flushing the feed line may thus result in a more homogenous fluid withdrawal.

FIGS. 13E and 13F illustrate steps of mixing the sample within the automated cell engineering system 600. As shown in FIG. 13E, a mixing sample is withdrawn from the automated cell engineering system 600. In embodiments, a specified volume is withdrawn for the mixing sample. In embodiments, the sterile sampling device 1300 may be oriented such that the distal end having the interconnect is facing upwards. This orientation causes the withdrawn sample to settle at a proximal portion of the sample reservoir of the sterile sampling device 1300, leaving a distal portion of the sample reservoir filled with gas.

Various orientations of the sterile sampling device 1300 are referred to with respect to the process shown in FIGS. 13A-13I. Sterile sampling device orientations may depend on specific applications. For example, fluid entering the sampling reservoir from the top to seed onto a scaffold within the sterile sampling device may yield a more optimal distribution of cells on the scaffold. Alternatively, orienting the sampling device such that fluid enters the sampling reservoir from the bottom may provide less mixing to prevent shear stresses on the fluid. Further, a preferred orientation may depend on the specific design of the sampling device. For example, for sterile sampling devices that rely on a filter at one end, it may be unsuitable to operate in an orientation that results in liquid contact with the filter. The orientations discussed herein are suitable for the operations and equipment discussed herein but may vary according to alternative requirements without departing from the scope of this disclosure.

After sample withdrawal, the sterile sampling device 1300 is used to inject the mixing sample back into the automated cell engineering system 600. FIG. 13F illustrates the mixing sample being injected back into the automated cell engineering system 600. In embodiments, the sterile sampling device 1300 is oriented such that the distal end having the interconnect is facing down to inject the mixing sample. This orientation causes the withdrawn sample to settle in a distal portion of the sample reservoir, leaving a proximal portion of the sample reservoir filled with gas. When the sample is pushed back into the automated cell engineering system, the remaining air in the sterile sampling device 1300 follows the sample and clears the feed line 1310. The steps of withdrawal and injection may optionally be repeated multiple times.

The steps of withdrawal and injection act to cause mixing in the system culture reservoir of the cassette 602 of the automated cell engineering system 600. Mixing the fluid within the system culture reservoir of the cassette 602 of the automated cell engineering system 600 may result in a more homogenous sample withdrawal. In further embodiments, the sample within the system culture reservoir of the cassette 602 of the automated cell engineering system 600 may be mixed in alternate ways, such as by rocking or shaking of the cassette 602 of the automated cell engineering system 600 or the system culture reservoir, via mechanical means included inside the cassette 602 of the automated cell engineering system 600, and any other suitable method.

FIG. 13G illustrates a step of withdrawing a sample from the cassette 602 of the automated cell engineering system 600. A specific volume of sample is withdrawn. In embodiments, the sterile sampling device 1300 may be oriented such that the distal end having the interconnect is facing up. This orientation causes the withdrawn sample to settle at a proximal portion of the sample reservoir of the sterile sampling device 1300, leaving a distal portion of the sample reservoir filled with gas.

This orientation may increase the ability to withdraw a specific sample volume. As the sample is withdrawn through the extension tube and the interconnect, it drops into the sample reservoir where it's volume can be easily ascertained by comparison with graded markings on the sterile sampling device.

FIG. 13H illustrates a step of injecting a portion of gas into the cassette 602 of the automated cell engineering system 600 after sample withdrawal. After sample withdrawal, the sterile sampling device 1300 may be oriented such that the interconnect is facing upwards. This orientation causes the sample to fall to the proximal end of the sample reservoir, leaving the distal portion of the sample reservoir filled with gas. The gas is then pushed back into the cassette 602 of the automated cell engineering system 600 system to clear the feed line 1310, as shown in FIG. 13H.

FIG. 13I illustrates a step of sample transfer. After withdrawal of the appropriate volume of sample, the sterile sampling device 1300 may be disconnected from the automated cell engineering system 600. The sample may be ejected from the sterile sampling device 1300 into an appropriate sample container for storage, shipping, analysis, or any further processing. In embodiments, the sample may remain in the sterile sampling device 1300 for storage prior to shipping, analysis, or further processing.

As described with respect to the method 1200 and the illustrated steps shown in FIGS. 13A-13I, a sterile sampling method consistent with embodiments herein may involve multiple sample transfers into and out of the automated cell engineering system 600. Thus, any contaminants introduced to the sterile sampling device have the potential to be introduced into the automated cell engineering system 600. Such contamination could damage or destroy a cell culture or other material being processed in the automated cell engineering system 600. The sterile sampling devices discussed herein, which include sterile sealing apparatuses, are employed to maintain the sterility of the sampling device throughout the sampling process. Sterility maintenance of the sample reservoir of the sterile sampling device prevents contamination of the sample reservoir when the sterile sampling device is operated to intake and expel samples. The sterility maintenance provided by the sterile sampling device maintains the sterility of any samples that are drawn into the sterile sampling device, whether they be mixing samples intended for return to the automated cell engineering system 600 or samples intended for later processing. The sterility maintenance described herein thus serves to maintain the sterility of the automated cell engineering system 600 and of any samples withdrawn for later processing.

### Exemplary Embodiments

Additional embodiments include the following.

Embodiment 1 is a sterile plunger syringe comprising: a syringe barrel defining a syringe reservoir and having an interconnect at a distal end and an opening surrounded by a syringe barrel flange at a proximal end; a syringe plunger including a syringe plunger rod flange, a plunger rod, and a reservoir face; a gasket disposed over the reservoir face and configured to provide a seal between the reservoir face and the syringe reservoir when the syringe plunger is seated within the syringe barrel; and a syringe plunger sealing device secured to the syringe barrel and configured to provide a syringe plunger seal.

Embodiment 2 is the sterile plunger syringe of embodiment 1, wherein the syringe plunger sealing device is secured to a proximal portion of the syringe barrel.

Embodiment 3 is the sterile plunger syringe of embodiment 1, wherein the syringe plunger sealing device is secured to the syringe barrel flange.

Embodiment 4 is the sterile plunger syringe of embodiment 3, wherein the syringe plunger sealing device is secured via an adhesive, a heat bonding, a chemical bonding, or a sonic welding.

Embodiment 5 is the sterile plunger syringe of any of embodiments 1-3, wherein the syringe plunger sealing device is removably secured via clamping.

Embodiment 6 is the sterile plunger syringe of any of embodiments 1-5, wherein the syringe plunger sealing device includes a plurality of accordion folds.

Embodiment 7 is the sterile plunger syringe of any of embodiments 1-6, wherein the syringe plunger sealing device seals the opening at the proximal end of the syringe reservoir.

Embodiment 8 is the sterile plunger syringe of any of embodiments 1-7, wherein the syringe plunger seal is substantially fluid-tight.

Embodiment 9 is the sterile plunger syringe of embodiment 8, wherein the syringe plunger seal is substantially gas-tight.

Embodiment 10 is a method of sterile sampling from an automated cell engineering system, the method comprising: providing a sterile plunger syringe including a syringe barrel, a syringe plunger, and a syringe plunger sealing device providing a syringe plunger seal; connecting the sterile plunger syringe to the automated cell engineering system; and withdrawing a biological sample from the automated cell engineering system with the sterile plunger syringe.

Embodiment 11 is the method of embodiment 10, wherein the syringe barrel defines a syringe reservoir having an interconnect at a distal end and an opening surrounded by a syringe barrel flange at a proximal end, the syringe plunger includes a syringe plunger rod flange, a plunger rod, a reservoir face, and a gasket disposed over the reservoir face and configured to provide a seal between the reservoir face and the syringe reservoir when the syringe plunger is seated within the syringe barrel, and the syringe plunger sealing device secured to the syringe barrel and configured to provide the syringe plunger seal.

Embodiment 12 is the method of embodiment 11, wherein the syringe plunger sealing device seals the opening at the proximal end of the syringe reservoir.

Embodiment 13 is the method of any of embodiments 10-12, further comprising: prior to connecting the sterile plunger syringe to the automated cell engineering system, introducing a portion of gas into the sterile plunger syringe.

Embodiment 14 is the method of any of embodiments 10-13, wherein the syringe plunger seal maintains sterility of an interior of the syringe reservoir.

Embodiment 15 is the method of any of embodiment 14, wherein maintaining sterility of the interior of the syringe reservoir prevents contamination of the syringe reservoir when the sterile plunger syringe is operated.

Embodiment 16 is the method of any of embodiments 10-15, further comprising injecting a portion of gas to clear a feed line within the automated cell engineering system after withdrawing the biological sample.

Embodiment 17 is the method of any of embodiments 10-16, further comprising injecting a portion of gas to clear a feed line within the automated cell engineering system before withdrawing the biological sample.

Embodiment 18 is the method of any of embodiments 10-17, further comprising pumping the sterile plunger syringe to cause mixing within the automated cell engineering system prior to withdrawing the biological sample.

Embodiment 19 is the method of embodiment 17, wherein pumping the sterile plunger syringe includes: withdrawing a mixing sample from the automated cell engineering system into the sterile plunger syringe; and returning the mixing sample to the automated cell engineering system.

Embodiment 20 is a method of sterile sampling from an automated cell engineering system, the method comprising: providing a sterile sampling device including a sample chamber defining a sample reservoir, a filling device, and a sterile sealing apparatus; connecting the sterile sampling device to a cassette of the automated cell engineering system; and withdrawing a biological sample from the automated cell engineering system via the filling device.

Embodiment 21 is the method of embodiment 20, further comprising: prior to connecting the sterile sampling device to the cassette, introducing a portion of gas into the sterile sampling device.

Embodiment 22 is the method of any of embodiment 20 or 21, further comprising maintaining sterility of the sample reservoir during withdrawing the biological sample via the sterile sealing apparatus.

Embodiment 23 is the method of embodiment 22, wherein maintaining sterility of the interior of the sample reservoir prevents contamination of the sample reservoir when the sterile sampling device is operated.

Embodiment 24 is the method of any of embodiments 20-23, further comprising injecting a portion of gas to clear a feed line within the automated cell engineering system after withdrawing the biological sample.

Embodiment 25 is the method of any of embodiments 20-24, further comprising injecting a portion of gas to clear a feed line within the automated cell engineering system before withdrawing the biological sample.

Embodiment 26 is the method of any of embodiments 20-25, further comprising causing mixing within the automated cell engineering system prior to withdrawing the biological sample via the filling device.

Embodiment 27 is the method of embodiment 26, wherein the mixing is caused by: withdrawing a mixing sample from the automated cell engineering system into the sample reservoir; and returning the mixing sample to the automated cell engineering system.

It will be readily apparent to one of ordinary skill in the relevant arts that other suitable modifications and adaptations to the methods and applications described herein can be made without departing from the scope of any of the embodiments.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

1. A sterile plunger syringe comprising:
a syringe barrel defining a syringe reservoir and having an interconnect at a distal end and an opening surrounded by a syringe barrel flange at a proximal end;
a syringe plunger including a syringe plunger rod flange, a plunger rod, and a reservoir face;
a gasket disposed over the reservoir face and configured to provide a seal between the reservoir face and the syringe reservoir when the syringe plunger is seated within the syringe barrel; and
a syringe plunger sealing device secured to the syringe barrel and configured to provide a syringe plunger seal.

2. The sterile plunger syringe of claim 1, wherein the syringe plunger sealing device is secured to a proximal portion of the syringe barrel.

3. The sterile plunger syringe of claim 1, wherein the syringe plunger sealing device is secured to the syringe barrel flange.

4. The sterile plunger syringe of any of claims 1-3, wherein the syringe plunger sealing device is secured via an adhesive, a heat bonding, a chemical bonding, or a sonic welding.

5. The sterile plunger syringe of any of claims 1-3, wherein the syringe plunger sealing device is removably secured via clamping.

6. The sterile plunger syringe of any of claims 1-5, wherein the syringe plunger sealing device includes a plurality of accordion folds.

7. The sterile plunger syringe of any of claims 1-6, wherein the syringe plunger sealing device seals the opening at the proximal end of the syringe reservoir.

8. The sterile plunger syringe of any of claims 1-7, wherein the syringe plunger seal is substantially fluid-tight.

9. The sterile plunger syringe of claim 8, wherein the syringe plunger seal is substantially gas-tight.

10. A method of sterile sampling from an automated cell engineering system, the method comprising:
providing a sterile plunger syringe including a syringe barrel, a syringe plunger, and a syringe plunger sealing device providing a syringe plunger seal;
connecting the sterile plunger syringe to the automated cell engineering system; and
withdrawing a biological sample from the automated cell engineering system with the sterile plunger syringe.

11. The method of claim 10, wherein the syringe barrel defines a syringe reservoir having an interconnect at a distal end and an opening surrounded by a syringe barrel flange at a proximal end, the syringe plunger includes a syringe plunger rod flange, a plunger rod, a reservoir face, and a gasket disposed over the reservoir face and configured to provide a seal between the reservoir face and the syringe reservoir when the syringe plunger is seated within the syringe barrel, and the syringe plunger sealing device secured to the syringe barrel and configured to provide the syringe plunger seal.

12. The method of claim 11, wherein the syringe plunger sealing device seals the opening at the proximal end of the syringe reservoir.

13. The method of any of claims 10-12, further comprising:
prior to connecting the sterile plunger syringe to the automated cell engineering system, introducing a portion of sterile gas into the sterile plunger syringe.

14. The method of any of claims 10-13, wherein the syringe plunger seal maintains sterility of an interior of the syringe reservoir.

15. The method of claim 14, wherein maintaining sterility of the interior of the syringe reservoir prevents contamination of the syringe reservoir when the sterile plunger syringe is operated.

16. The method any of claims 10-15, further comprising injecting a portion of gas to clear a feed line within the automated cell engineering system after withdrawing the biological sample.

17. The method of any of claims 10-16, further comprising injecting a portion of gas to clear a feed line within the automated cell engineering system before withdrawing the biological sample.

18. The method of any of claims 10-17, further comprising pumping the sterile plunger syringe to cause mixing within the automated cell engineering system prior to withdrawing the biological sample.

19. The method of claim 17, wherein pumping the sterile plunger syringe includes:
withdrawing a mixing sample from the automated cell engineering system into the sterile plunger syringe; and
returning the mixing sample to the automated cell engineering system.

20. A method of sterile sampling from an automated cell engineering system, the method comprising:
providing a sterile sampling device including a sample chamber defining a sample reservoir, a filling device, and a sterile sealing apparatus;
connecting the sterile sampling device to a cassette of the automated cell engineering system; and
withdrawing a biological sample from the automated cell engineering system via the filling device.

21. The method claim 20, further comprising:
prior to connecting the sterile sampling device to the cassette, introducing a portion of gas into the sterile sampling device.

22. The method of any of claim 20 or 21, further comprising maintaining sterility of an interior of the sample reservoir during withdrawing the biological sample via the sterile sealing apparatus.

23. The method of claim 22, wherein maintaining sterility of the interior of the sample reservoir prevents contamination of the sample reservoir when the sterile sampling device is operated.

24. The method any of claims 20-23, further comprising injecting a portion of gas to clear a feed line within the automated cell engineering system after withdrawing the biological sample.

25. The method of any of claims 20-24, further comprising injecting a portion of gas to clear a feed line within the automated cell engineering system before withdrawing the biological sample.

26. The method of any of claims 20-25, further comprising causing mixing within the automated cell engineering system prior to withdrawing the biological sample via the filling device.

27. The method of claim 26, wherein the mixing is caused by:
withdrawing a mixing sample from the automated cell engineering system into the sample reservoir; and
returning the mixing sample to the automated cell engineering system.
